(19) United States
(12) Patent Application Publication
(10) Pub. No.: US 2013/0129713 A1
(43) Pub. Date: May 23, 2013

(54) METHOD OF ANTIGEN LOADING FOR IMMUNOTHERAPY

(71) Applicants: Maria Rescigno, Milano (MI) (IT); Fabiana Saccheri, Milano (MI) (IT)

(72) Inventors: Maria Rescigno, Milano (MI) (IT); Fabiana Saccheri, Milano (MI) (IT)

(73) Assignee: IEO - ISTITUTO EUROPEO DI ONCOLOGIA S.R.L., Milano (MI) (IT)

(21) Appl. No.: 13/813,778

(22) PCT Filed: Aug. 4, 2011
(86) PCT No.: PCT/EP11/63440

§ 371 Date: Feb. 1, 2013

Related U.S. Application Data
(60) Provisional application No. 61/370,599, filed on Aug. 4, 2010.

(51) Int. Cl. A61K 39/00; A61K 39/395

(52) U.S. Cl. CPC A61K 39/0011; A61K 39/395
USPC 424/130.1; 435/373; 435/325; 424/277.1

(57) ABSTRACT

It is disclosed a method for obtaining a specific tumor antigen peptide repertoire loaded and/or activated dendritic cell comprising the steps of: a) exposing in suitable conditions a tumor cell expressing said specific tumor antigen peptide repertoire to at least one Pattern Recognition Receptor (PRR) agonist and/or to an inflammatory cytokine to obtain a tumor cell with an increased activity of at least one protein belonging to the group of connexins; b) co-culturing said tumor cell with an increased activity of at least one protein belonging to the group of connexins with dendritic cells, to get specific tumor antigen peptide repertoire loaded and/or activated dendritic cells; Step a) and b) are performed simultaneously or in sequence. The group of connexins preferably consists of connexin 43, connexin 40, connexin 45, connexin 47, connexin 50.

### TECHNICAL FIELD

Bacteria can be successfully used to induce tumor cell to dendritic cell communication via gap-junctions by upregulating the expression of connexins, preferably connexin 43 (encoded i.e. by Mus musculus: gene ID: 14609; Homo sapiens: gene ID: 2697) and/or connexin 40 (encoded i.e. by Mus musculus gene ID: 14613; Homo sapiens gene ID: 2702), and/or connexin 45, (encoded i.e. by Mus musculus gene ID: 14615; Homo sapiens gene ID: 10052) and/or connexin 47 (encoded i.e. by Mus musculus gene ID: 118454; Homo sapiens gene ID: 57165), and/or connexin 50 (encoded i.e. by Mus musculus gene ID: 14616 and Homo sapiens: gene ID: 2703) allowing cross-presentation of tumor antigens and effective activation of tumor specific immune responses in mice. Cross-presentation enables dendritic cells (DCs) to present exogenous antigens to CD8 T cells. A new mechanism of antigen cross-presentation that is based on peptide transfer from donor to acceptor cells via gap junctions (GJs) has been described, but its exploitation in tumor immunity has not been analyzed. Authors demonstrate here that by the use of bacteria it is possible to induce, in both human and murine melanoma cells, the upregulation of Connexin (Cx)43, a ubiquitous protein involved in GJ formation that is normally lost during melanoma progression. Bacteria-treated melanoma cells can establish functional GJs with adjacent DCs. These GJs allow the transfer of antigenic pre-processed peptides to DCs for T cell activation. Melanoma cells stably silenced for Cx43, when infected in vivo with bacteria, fail to elicit a cytotoxic antitumor response that controls the growth of distant untreated tumors, indicating that this mechanism is the principal one that is used in vivo for the generation of anti-tumor responses. This Connexin 43-dependent cross-presentation pathway is strikingly more effective than standard protocols of DC loading (peptide, tumor lysates or apoptotic bodies) for the generation of DC-based tumor vaccines that are protective in both a preventive and a therapeutic setting. In conclusion, authors exploited an anti-microbial response that has not undergone immune evasion pressure to activate CD8 T cells specific for tumor-generated peptides for direct recognition and killing of tumor cells.

Dendritic cells (DCs) are key players in the activation of T cells (1). DCs comprise a family of antigen presenting cells, including plasmacytoid and conventional (myeloid) DCs. DCs are endowed with the ability to present exogenous antigens that have not been generated within DCs for the activation of T cells, via the cross-presentation pathway. Cross-presentation is required for the initiation of effective anti-tumor T cell responses (2) and the repertoire of presented peptides is crucial to activate T cells that will recognize and kill tumor cells (3). However, the antigen presentation machinery, and in particular the proteasome, differs between tumor cells and dendritic cells (4). A major drawback is that DCs could process and present peptides that are different from those presented by tumor cells, thus initiating a tumor-specific response that will not recognize the tumor (4).

Gap junctions (GJs) are channels that connect the cytoplasm of two adjacent cells (5). They allow the transfer of small molecules including ions, second messengers and metabolites up to 1 kDa (5). GJ intercellular communication (GJIC) has been shown to participate to many physiological events like cell cycle control, differentiation, cell synchronization and metabolic coordination (5, 6). GJs are formed by two hemichannels, called connexons, each made of six Connexin proteins. There are at least 21 Connexins most of which are tissue specific except for Connexin (Cx) 43 that is ubiquitously expressed (7). Loss of GJIC is a common feature in many human tumors and can occur early during tumorigenesis (8, 9). Recently, GJs have been shown to play a prominent role also in the immune system (7). They are required for B and T cell differentiation, antibody secretion by B cells, T regulatory cell activity (10) and dendritic cell activation (11, 12). GJs are also involved in antigen cross-presentation by allowing the spreading of small linear peptides (up to 16 amino acid long) between neighboring cells (13), including apoptotic cells (14). The transferred peptides can be loaded onto MHC class I molecules and be presented on the surface of acceptor cells. This mechanism may be used to favor the killing of a ring of non-infected cells surrounding the infected tissue, and participate to ‘sanitation’ of the tissue. In addition, it may be used by the immune system to activate T cells specific for the infectious agent, via transfer of antigenic peptides from infected cells to non-infected professional antigen presenting cells (13). This pathway is also involved in killing of endothelial cells by tumor-specific cytotoxic T cells (CTL)s after transfer of peptides from tumor cells to endothelial cells in vitro (15). However, the in vivo relevance of GJ-cross-presentation pathway and its exploitation in anti-tumor immunity has not been addressed.

The use of bacteria as anticancer agents has long been proposed (16). The Gram-negative bacteria, as Salmonella typhimurium, are particularly appealing for their ability to home preferentially to tumor sites (17). Salmonella can be used as a delivery vector for cytokines (18), chemokines (19), tumor antigens (20) and DNA-based vaccines (21, 22). Authors recently showed that the intratumoral injection of S. typhimurium allows breaking ignorance and tolerance to melanoma. It acts both locally by recruiting immune cells and leading to the elimination of the treated mass (23), and systemically favoring the development of an antitumor response via the cross-presentation of tumor antigens (24).

In this study, authors analyzed how Gram-negative bacteria, i.e. Salmonella typhimurium infection facilitated the cross-presentation of tumor antigens and its exploitation to generate potent DC-based tumor vaccines.

### PRIOR ART

Dillman R. et al (Cancer Biotherapy & Radiopharmaceuticals, 2009, 24, p. 311) discloses an autologous tumor cell vaccines consisting of dendritic cells (DCS), derived from patient's peripheral blood cells cultured in (IL)-4 and granulocyte macrophage colony-stimulating factor (GM-CSF), which had phagocytosed irradiated autologous tumor cells from a continuously proliferating, self-renewing, autologous tumor cell (TC) culture.

WO2009/040413 discloses a process to obtain activated antigen-presenting cells that are useful for therapies against cancer and immune system-related diseases, by means of a cellular composition that contributes to stimulate the activated antigen-presenting cells to induce specific immune response against tumours. The method induces the differentiation of monocytes in APC (dendritic cells) by stimulation in culture using cytokines, growth factors and/or mixture of lysate or extracts of tumour cells. Mendoza-Naranjo A, et al, and Salazar-Onfray, J. Immunol. 2007; 178;6949-6957 describes the use of melanoma cell lysate stimulated with TNFalfa to induce gap junctions to promote Ag transfer between ex vivo produced hDCs from melanoma patients.

The instant invention refers to the activation of DC cells by means of specific antigen cross-presentation, the antigen being processed by the tumour cell itself and vehiculated to DC cells through gap junction.

WO2008019366 disclose methods and compositions for increased priming of T-cells through cross-presentation of exogenous antigens. It refers to particles (S.Cerevisiae) on the surface of which the antigen is attached, and administering the antigen preparation to the animal, wherein the particles are taken up by antigen presenting cells (APC) of the animal via phagocytosis.

The instant invention refers to a vehiculation of the tumor antigen to DC cells through gap junction.

US 20090324651 discloses methods for stimulating an immune response using bacterial antigen delivery system. It relates to the use of the type III secretion system of bacteria to stimulate immune responses against tumor antigen(s) for treating antigen-loss variant tumors. Methods are provided for stimulating and/or increasing an immune response against tumor antigens.

The prior art document also relates to the preparation of antigen presenting cells from peripheral blood mononuclear cells using bacteria having a type III secretion system. The method refers to the culture of PBMCs, previously contacted with an avirulent bacteria (such as S. typhimurium) expressing a tumor antigen, and isolating antigen presenting cells. Salmonella acts as vehicle of the tumoral antigen (previously “loaded” on bacteria) to the APC cell (degradation of antigen is still made by the APC).

Eugenin E. A. et al, and Juan C. Saez. J. Immunol 2003, 170:1320-1328 discloses that TNF-alfa plus IFN-gamma induce connexin 43 expression and formation of gap junctions between human monocytes and macrophages.

The instant invention refers to gap junction and transfer of tumour antigen between tumour cells and DC-cells.

Elgueta R. et al, and Saez J. J Immunol 2009, 183(1):277-84 reports the formation of gap junctions between DCs and T cells and their role on T cell activation during Ag presentation by DCs.

The instant invention refers to the antigen loading of dendritic cells by means of gap junctions formation between dendritic and tumoral cells.

WO2004/050855 discloses a one-step method for producing antigen loaded dendritic cells vaccine comprising an activator such as TNF alpha preferably in combination with at least one growth factor such as GM-CSF and at least one soluble or particulate antigen.

In the instant invention, the antigen is processed by the tumour cell itself and vehiculated to DC cells through gap junction.

### DESCRIPTION OF THE INVENTION

In the instant invention any inducer of connexins, preferably of the connexin 43 and/or 40 and/or 45 and/or 47 and/or 50 gap junction protein, i.e. pattern recognition receptor (PRR) agonists, preferably Gram-negative bacteria or components thereof, and/or inflammatory cytokines, preferably gamma-IFN (IFN-g), is used to create cell-cell communication through gap junctions, for the transfer of tumor antigen between tumor cell and dendritic cell; in this case, the antigen is processed directly by the tumor cells.

It is an object of the instant invention a method for obtaining a specific tumor antigen peptide repertoire loaded and/or activated dendritic cell comprising the steps of:
a) exposing in suitable conditions a tumor cell expressing said specific tumor antigen peptide repertoire to at least one Pattern Recognition Receptor (PRR) agonist and/or to one inflammatory cytokine to obtain a tumor cell with an increased activity of at least one protein belonging to the group of connexins;
b) co-culturing said tumor cell with an increased activity of at least one protein belonging to the group of connexins with dendritic cells, to get specific tumor antigen peptide repertoire loaded and/or activated dendritic cells;
   wherein step a) and b) are performed simultaneously or in sequence.

Tumor cells present specific tumor antigen peptide repertoire derived either from tumor associated antigens or by proteins expressed also in non tumor cells that are specifically cleaved in the tumor cell, as i.e. described in Mocellin S, Mandruzzato S, Bronte V, et al. Part I: Vaccines for solid tumours. Lancet Oncol 2004;5:681-9.

An antigen loaded DC is a well known definition for the skilled person, and refers also to antigen degradation and peptide loading onto MHC molecules occurring intracellularly in Antigen Presenting Cells (APCs, such as dendritic cells). CD8+and CD4+ T cells expressing clonally distributed receptors recognize fragments of antigens (peptides) associated with MHC class I and II molecules, respectively (Guermonprez P, Valladeau J, Zitvogel L, et al. Antigen presentation and T cell stimulation by dendritic cells. Annu Rev Immunol 2002;20:621-67).

As to the meaning of activated DCs, a well known definition for the skilled person, the dendritic cell matures into a highly effective antigen-presenting cell (APC) and undergoes changes that enable it to activate antigen-specific lymphocytes that it encounters i.e. in the lymph node. Activated dendritic cells secrete cytokines that influence both innate and adaptive immune responses (Immunobiology: The Immune System in Health and Disease. 5th edition. Janeway C A Jr, Travers P, Walport M, et al. New York: Garland Science; 2001).

Pattern recognition receptors (PRRs) refer to germline-encoded receptors that recognize molecular structures that are broadly shared by pathogens, known as pathogen-associated molecular patterns (PAMPs, Kawai T, Akira S. Toll-like receptors and their crosstalk with other innate receptors in infection and immunity. Immunity 2011;34:637-50).

A PRR agonist refers to a compound (either natural or synthetic) that binds to PRR and triggers a response.

Dendritic cells present preprocessed tumor-derived peptide without the need of phagocytosis; since DCs have a different proteasome than tumor cells, they may generate peptides that are not readily produced within tumor cells. Hence, tumor cells do not present the peptides to which cytotoxic T cells are specific. A GJ-dependent mechanism of cross-presentation that instead is based on pre-processing of antigenic peptides within tumor cells may overcome this drawback.

In the context of the present invention, increased activity of at least one protein belonging to the group of connexins means increased amount of at least one protein belonging to the group of connexins and/or increased transfer of material from one donor cell to an acceptor cell also known as intercellular communication.

In a preferred embodiment the group of connexins consists of: connexin 43, connexin 40, connexin 45, connexin 47 and connexin 50 and orthologous, allelic variants or isoforms thereof.

In a preferred embodiment the inflammatory cytokine is gamma-IFN.

In a preferred embodiment the method further comprises step c): purifying said specific tumor antigen peptide repertoire loaded and/or activated dendritic cells.

According to a preferred aspect of the invention, the tumor cell is an established tumor cell line, or a combination of tumor cell lines expressing a specific tumor antigen peptide repertoire or a tumor cell isolated by a tumor affected subject.

The tumor cell derives from solid or non-solid tumors, including but not limited to melanoma, lung carcinoma, colorectal adenocarcinoma, prostate adenocarcinoma, leukemia and T cell-lymphoma and the said specific tumor antigen peptide repertoire is specific for said tumor.

Dendritic cells may be autologous or HLA-compatible or -semi-compatible allogenic dendritic cells.

In a preferred aspect PRR agonists are Gram-negative, Gram-positive bacteria or components thereof. Preferably Gram negative bacteria belong to the Salmonella genus, more preferably to non virulent strains of Salmonella genus; in a further preferred embodiment Gram negative bacteria components are LPS and/or flagellin. Alternatively Gram positive bacteria components are LTA.

It is a further object of the invention a specific tumor antigen peptide repertoire loaded and/or activated dendritic cell obtainable according to the method above disclosed.

The specific tumor antigen peptide repertoire loaded and/or activated dendritic cell of the invention may be advantageously used as a medicament, preferably as a tumor immunotherapeutic agent or as tumor vaccine, also in in combination with antibodies and/or chemotherapeutics.

The specific tumor antigen peptide repertoire loaded and/or activated dendritic cell of the invention may be administered to a subject in suitable amounts by conventional administration routes, such as intradermal, also at multiple administration dosages, i.e. at weekly intervals, for tumor treatments.

It is a further object of the invention the use of specific tumor antigen peptide repertoire loaded and/or activated dendritic cell of the invention to generate specific CTLs by means of their incubation with a sample of CD8+ T cells isolated from a donor.

It is a further object of the invention the use of at least one PRR agonist and/or one inflammatory cytokine for increasing the activity of at least one protein belonging to the group of connexins in a tumor cell expressing a specific tumor antigen peptide repertoire.

The present invention will be described through non-limitative examples, with reference to the following figures:

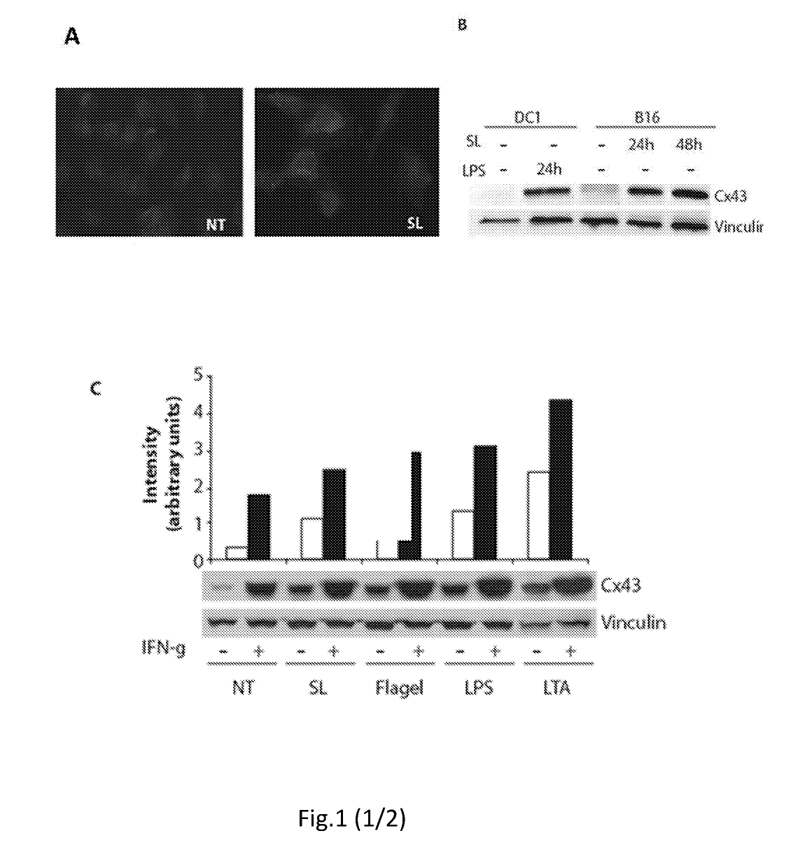

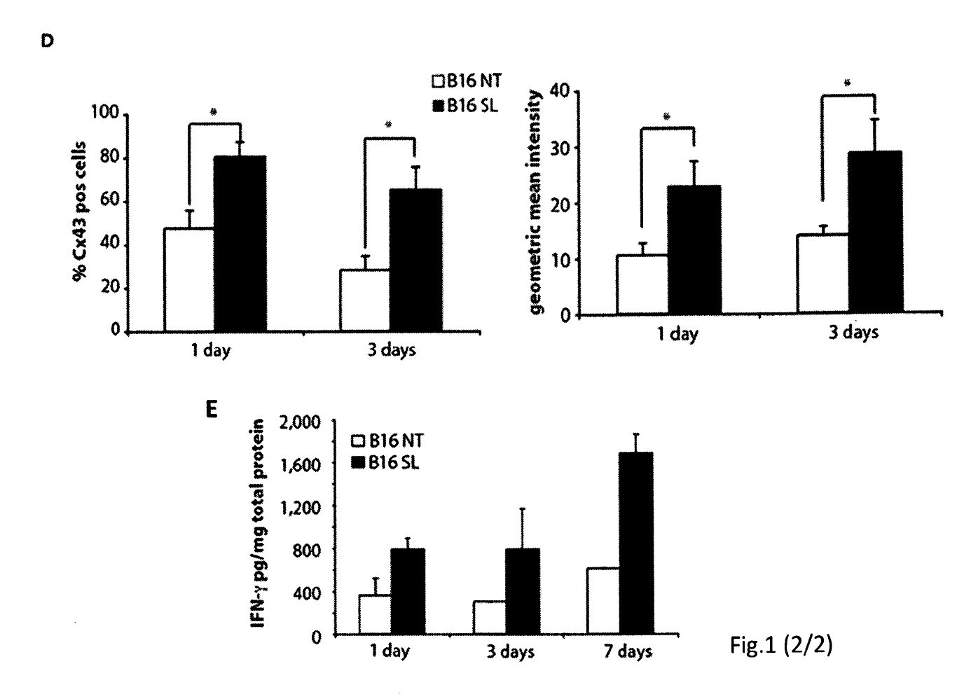

FIG. 1. Bacteria and Interferon (IFN)-g . upregulate Connexin (Cx) 43 expression in tumor cells. (A) B16 cells were left untreated (NT) or incubated with bacteria (Salmonella, SL) for 2 h. After 24h Cx43 expression was evaluated by immunofluorescence (Cx43: red; Dapi: blue). (B) B16 cells or DC1 dendritic cells were left untreated, incubated with bacteria (Salmonella, SL) for 2 h or lypopolysaccharide (LPS) for 24 h. Western blot after 24 and 48 h using anti-Cx43 or anti-vinculin antibody is shown. (C) B16 cells were left untreated (NT), incubated with Salmonella (SL) or bacterial products (Flagellin, Flagel; lypopolysaccharide, LPS; Lipoteichoic acid, LTA) in the presence or absence of IFN-g. Western blot after 24 h using anti-Cx43 or anti-vinculin antibody is shown. Graph bars show band quantification in the presence (black bars) or absence (white bars) of IFN-g. (D-E) B16 established tumors were treated (B16 SL, black bars) or not (B16 NT, white bars) with Salmonella (SL). (D) 1 and 3 days later mice were sacrificed, tumors were smashed and cells analyzed for Cx43 expression by FACS. Percentage of Cx43⁺ cells (left) and geometric mean intensity (right) are shown. (E) 1, 3 and 7 days after SL infection, mice were sacrificed, tumors were smashed and IFN-g production was measured by ELISA. Error bars: s.d. Each experiment was repeated 3-4 times with similar results.

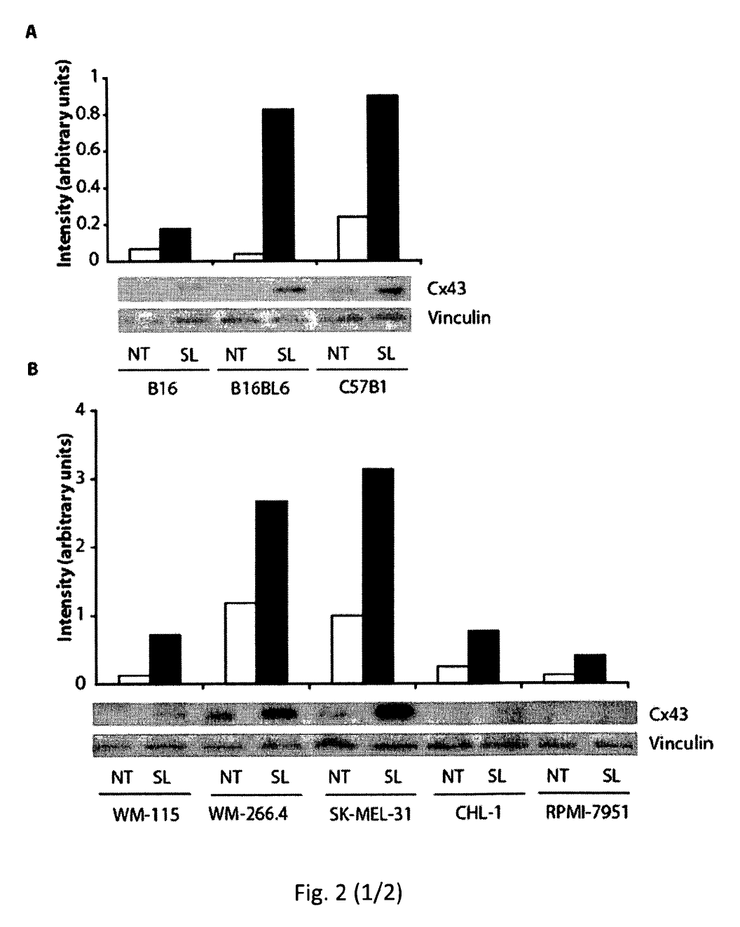

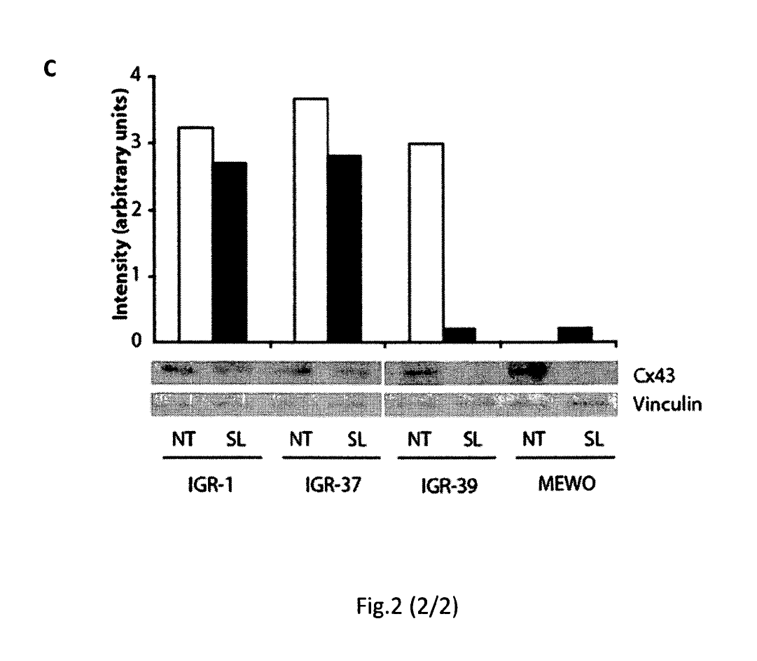

FIG. 2. In all murine and those human melanoma cell lines that do not express Cx43, it is upregulated in response to Salmonella. Murine (A) and human (B-C) melanoma cell lines were incubated (SL) or not (NT) with Salmonella in medium without antibiotics for 2 h. 24 h later, cells were analyzed for Cx43 expression by Western blot. Vinculin was used as reference protein. Bars represent the quantification of the bands of cells treated (black bars) or not (white bars) with Salmonella. The intensity of the band is shown as arbitrary units.

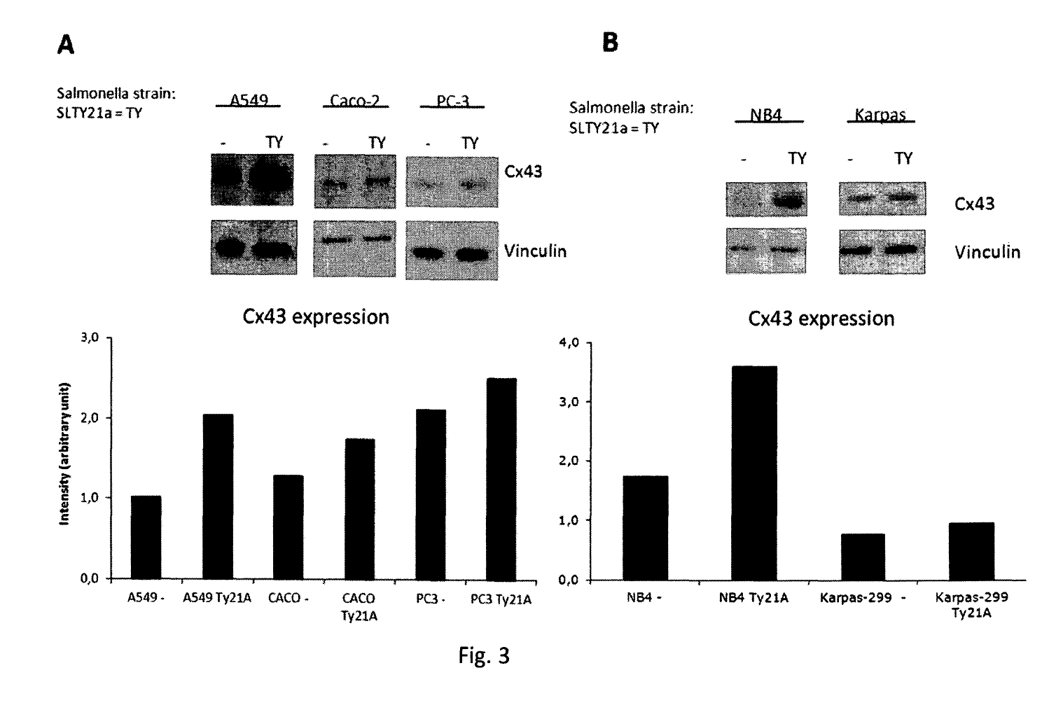

FIG. 3. Salmonella infection induces up-regulation of Cx43 in several human tumor cell lines. Upregulation of Cx43 was evaluated in human tumor cells (A, solid; B, non solid tumors) after infection Salmonella typhi (SLTY21a). Western blot analysis after 24h using anti-Cx43 or anti-vinculin antibody is shown. Graph bars show band quantification, normalized versus vinculin.

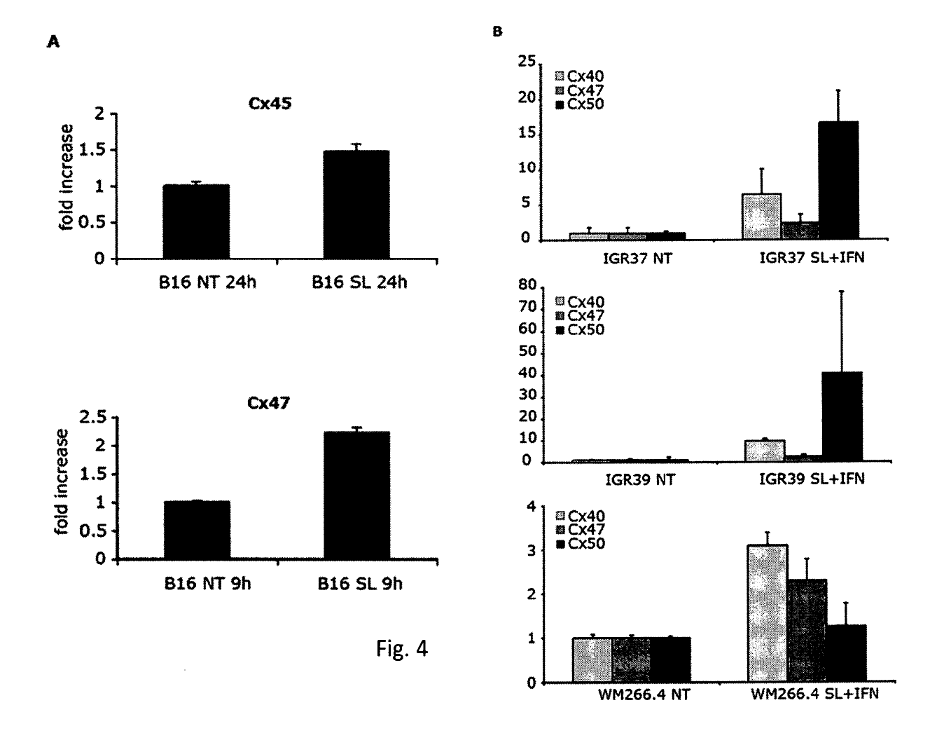

FIG. 4. Salmonella modulates also other connexins both in mouse and human melanoma cells. mRNA expression of connexins 45, 47 in B16 cells (a) and connexins 40, 47, and 50 in human melanoma cell lines IGR37, IGR39 and WM266.4 (b) is shown. (a) The expression was evaluated by Sybr Green assay on Salmonella (SL)-treated or not (NT) B16 cells 9 and 24h after the infection. (b) The expression was evaluated by Sybr Green assay on cells treated with Salmonella in combination with IFN-g (SL+IFN) or left untreated (NT). The expression of the genes of interest is represented as fold increase of the expression compared to not treated melanoma cells after normalization to TBP.

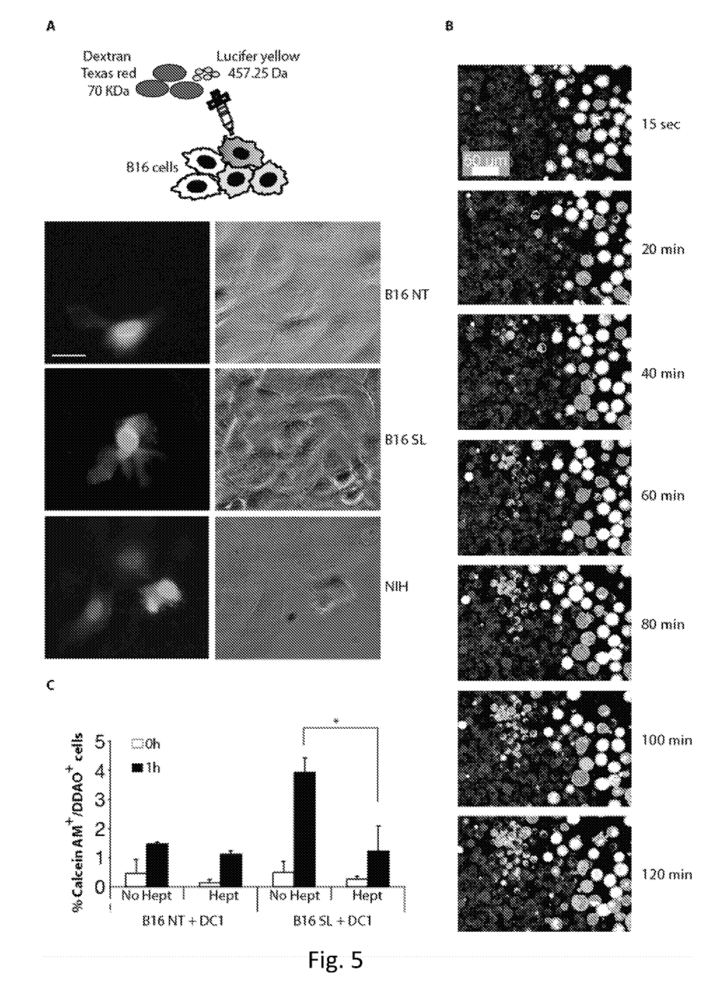

FIG. 5. Bacteria-induced upregulation of Cx43 expression correlates with establishment of functional gap junctions. (A) B16 cells were infected (B16 SL) or not (B16 NT) in vitro with Salmonella and after 24 h were microinjected with a mix of the GJ diffusible dye Lucifer yellow (green) and the non-transferable dye Dextran Texas red (red, to mark microinjected cells). Cells were fixed immediately after microinjecting the last cell and observed for dye transfer by fluorescence microscopy. Untreated NIH-3T3 cells were used as positive control. Magnification: 40X; scale bar: 20 mm. (B) Confocal microscopy analysis. B16 cells were infected with Salmonella and after 24 hours were stained with calcein-AM (in green/white), while DCs, previously treated with LPS for 1 hour, were stained with DDAO (in red). A drop of each population was plated onto a microscope slide close to each other, and the cells were co-incubated for 1 hour. The cells were analyzed by confocal microscopy for 2 hours. Representative single frames extrapolated from the movie every 20 minutes are shown. (C) Only Salmonella infected cells can communicate with DCs via a GJ-dependent mechanism. B16 cells were infected or not with Salmonella, pulsed with the GJ-diffusible dye Calcein-AM and incubated with DDAO-labeled DCs, in the presence or absence of heptanol. One h later the % of cells double positive for DDAO and Calcein-AM, representing the DCs that received the dye from tumor cells, was evaluated by FACS (Black bars). Error bars, s.d. *, p<0.05. Each experiment was repeated twice with similar results.

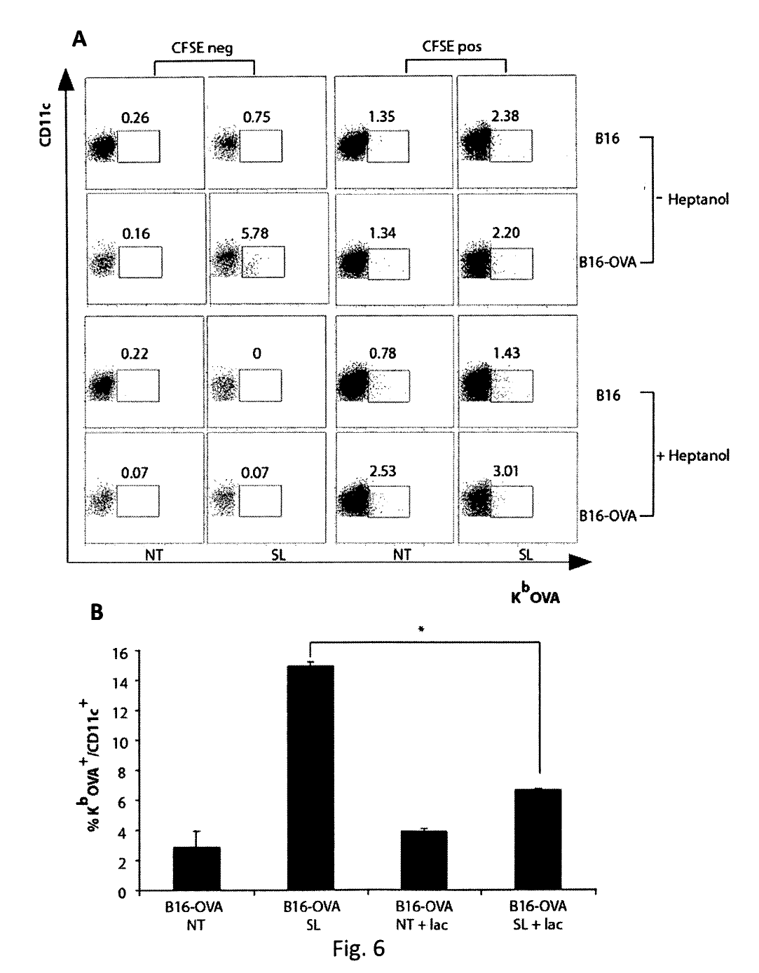

FIG. 6. Bacterial infection facilitates cross-presentation of preprocessed tumor associated antigens through gap junctions. (A) B16 or B16-OVA cells were infected (SL) or not (NT) with Salmonella. 24 h later they were stained with CFSE and incubated with mature DCs in the presence or absence of heptanol for 24 h. Dot plots show cells positive for CD11c and K^{b}OVA in the CFSE⁻ (left) and CFSE⁺ gate (right). Numbers show the percentage of CD11c K^{b}OVA double positive cells in the gate. One representative experiment of three is shown. (B) Preprocessing in tumor cells is required for effective cross-presentation. B16-OVA cells were treated as above but in the presence or absence of the irreversible proteasome inhibitor lactacystin (lac). The percentage of K^{b}OVA/CD11c double positive cells is shown. Error bars: s.d. *, p<0.05. One experiment representative of two is shown. The % of CD11c⁺K^{b}OVA⁺ cells varied between experiments (compare A to B) but the trend was always very similar.

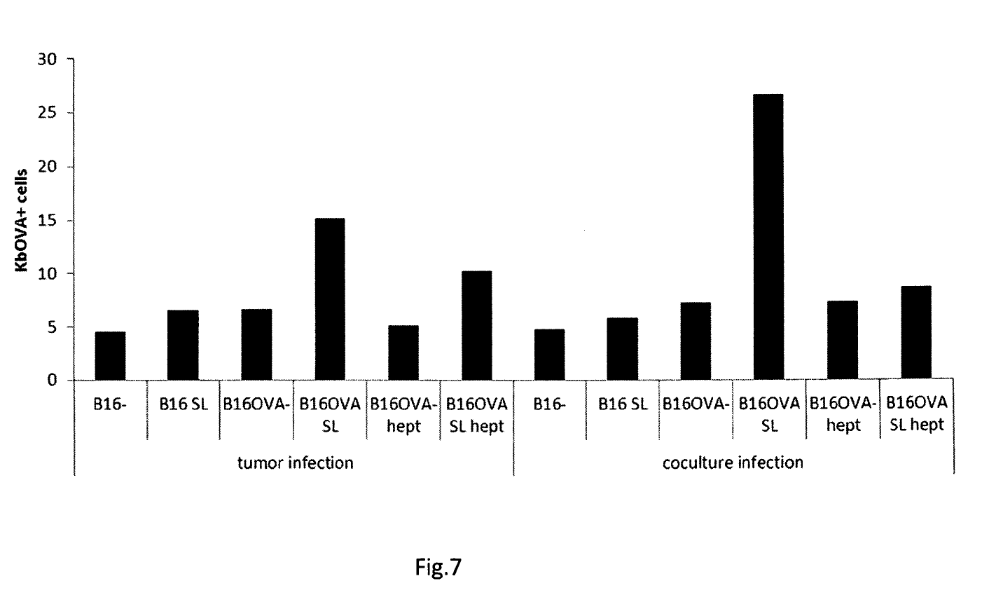

FIG. 7. OVA peptide presentation after infection of tumor-DCs coculture. Comparison of OVA-presentation in case of infection of B16-OVA alone (tumor infection) prior to incubation with DCs or B16-OVA-DCs coculture (coculture infection) shows that they are both effective in inducing antigen presentation by DCs and when B16-OVA and DCs were infected together in coculture, OVA presentation was even more efficient. As a negative control B16 cells not expressing OVA were used. The experiment was done in presence or absence of Heptanol (Hept).

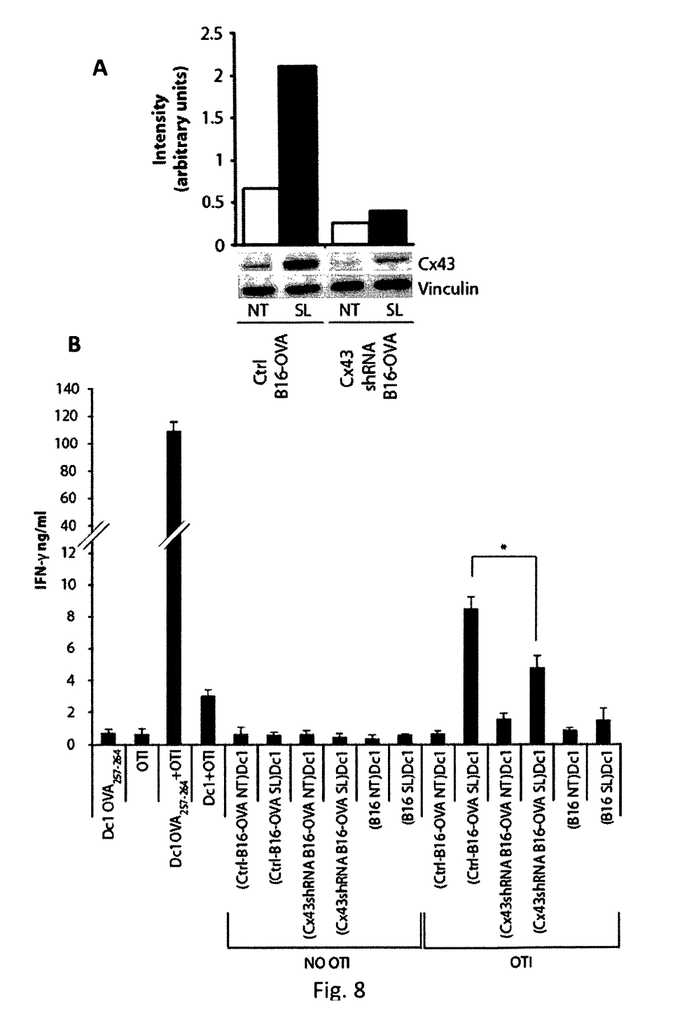

FIG. 8. Silencing of Cx43 in B16-OVA cells strongly reduces OVA cross-presentation. (A) Effective silencing of Cx43 in B 16-OVA was assessed by Western blot analysis. Bars represent the quantification of Cx43 with (black bars) or without (white) Salmonella treatment, expressed in arbitrary units. (B) Control shRNA ineffective in silencing Cx43 in B16OVA (ctrl-B16-OVA) and Connexin 43-interfered B16-OVA (Cx43 shRNAB 16-OVA) were infected with Salmonella and were co-cultured with mature dendritic cells in a 1:1 ratio. After 24 hours dendritic cells were purified by CD11c positive selection and then treated with 25 μg/ml Mitomycin C in order to arrest the proliferation of possible remaining tumor cells. Cells were extensively washed and incubated in the presence (OTI) or absence (NO OTI) of OTI CD8 T cells with and without the OVA₂₅₇₋₂₆₄ peptide. After 48 hours the supernatant was collected and the amount of IFN-γ was assessed by ELISA. Controls were DCs pulsed or not with the OVA peptide and incubated or not with OTI T cells. The experiment was repeated twice in triplicate. Error bars represent standard deviation of the triplicates.

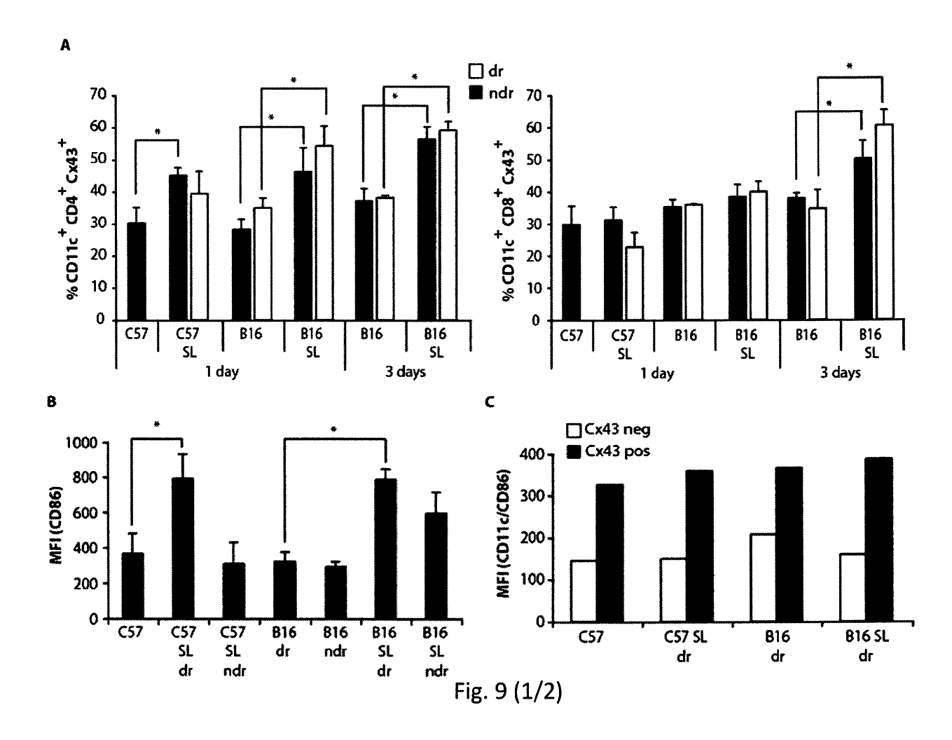

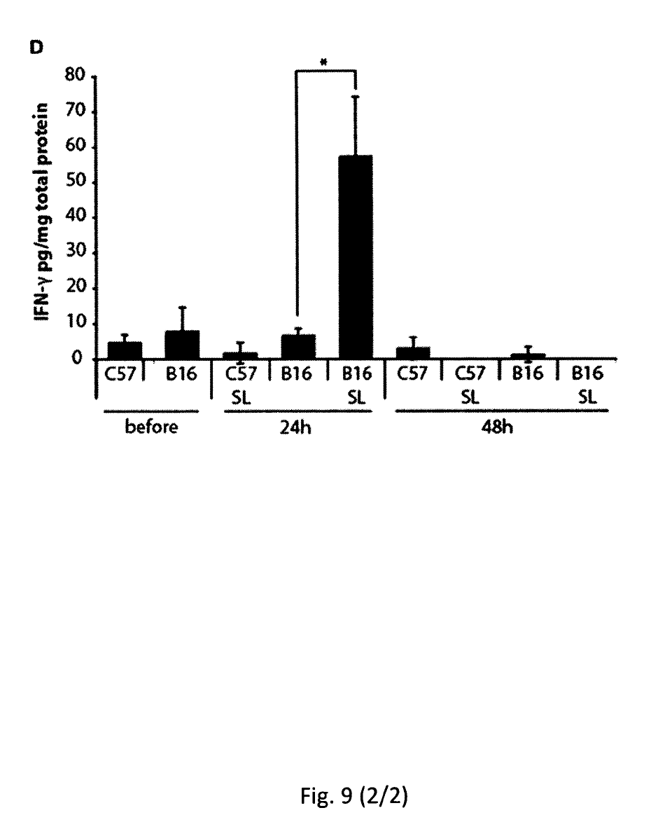

FIG. 9. Intratumoral bacterial injection increases the percentage of CX43⁺ DCs in lymph nodes. (A) Salmonella injected in the tumor (B16) or skin (C57) induces the increase of Cx43⁺ dendritic cells in draining lymph nodes. C57/BL6J mice were injected with B16 cells and after 10 days were intratumorally injected with Salmonella (SL) or PBS as a control. After 1 or 3 days draining (dr) and not draining (ndr) lymph nodes were collected and analyzed by FACS for the presence of CD11c⁺CD4⁺Cx43⁺ (left graph) and CD11c⁺CD8⁺Cx43⁺(right graph) cells. Error bars, s.d. *, p<0.05. (B-C) Upregulation of Cx43 in dendritic cells of draining lymph nodes correlates with their activated state. The experiment was performed as in A, then mean fluorescence intensity (MFI) of CD86 in CD11c⁺ cells (B) and the MFI of CD86 in CD11c⁺ cells in the Cx43 negative and positive fraction (C) was analyzed by FACS. Error bars: s.d. *, p<0.05. (D) Analysis by ELISA of IFN-g in the serum of the above described mice. Error bars: s.e. *,p<0.05.

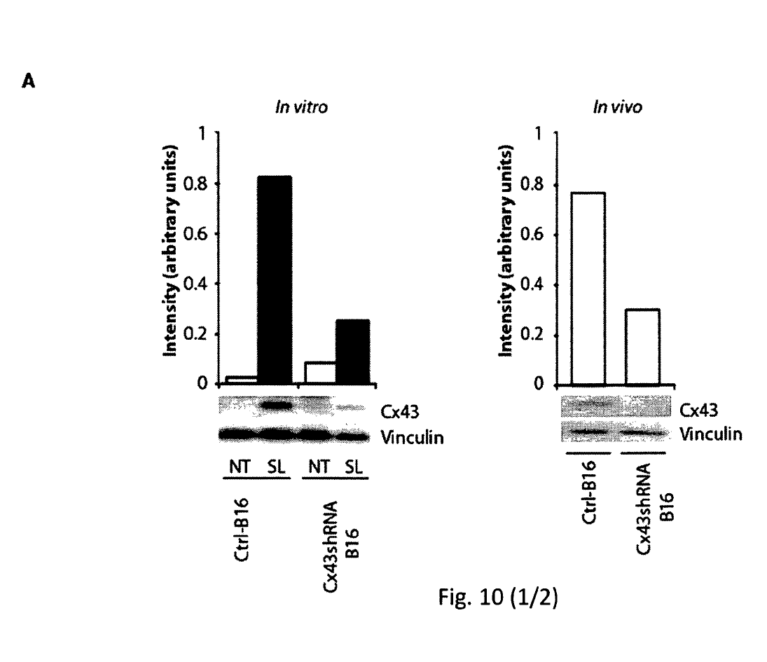

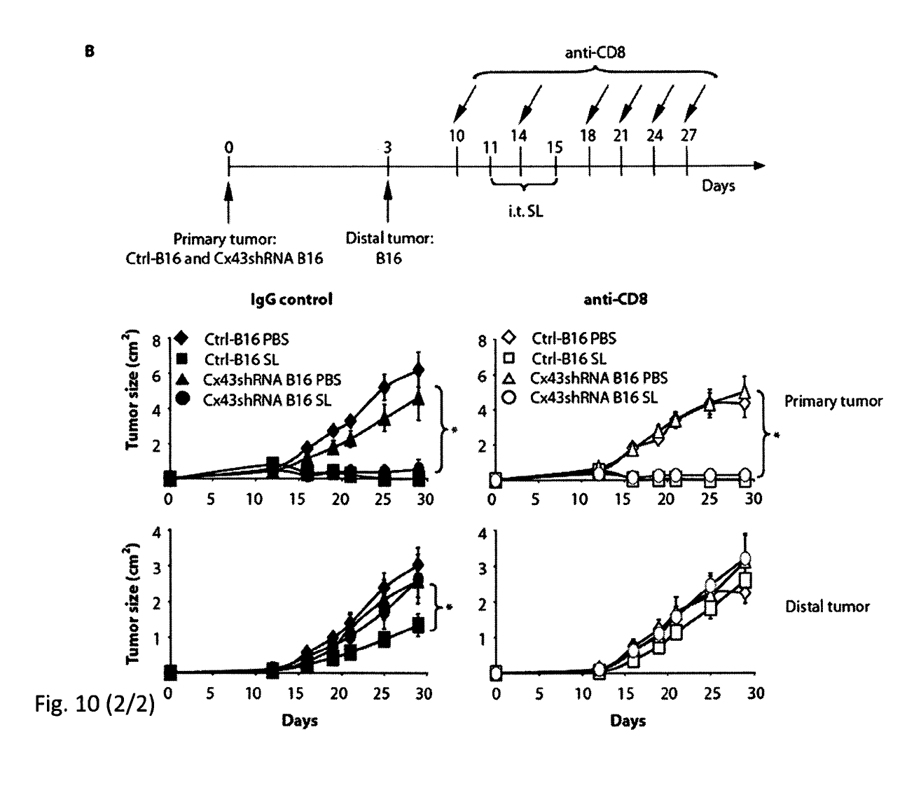

FIG. 10. Cx43 expression by tumor cells is required for the initiation of anti-tumor immunity. (A) Effective knock-down of Cx43 was determined by Western blot on control B16 (Ctrl-B16: using and shRNA ineffective in silencing Cx43) or on Cx43shRNA B16 cells infected (SL, black bars) or not (NT, white bars) with Salmonella in vitro (left) and on established B16Cx43shRNA tumors in vivo (right). Bars represent the quantification of Cx43 expressed in arbitrary units. (B) Mice received two tumor inoculations following the indicated schedule. B16 control or Cx43shRNAB16 cells were injected on the left flank for the generation of the primary tumor while the distal tumor was formed by B16 WT cells injected in the right flank. Bacteria or PBS were injected only in the primary tumor. Groups of 8 mice were injected i.p. with neutralizing anti-CD8 (right graphs) or isotype control (left graphs) antibody at the days indicated in the schedule. The growth of the tumors is shown over time (upper graphs: primary tumors; lower graphs: distal tumors). Error bars, s.e. *, p<0.05 when comparing the growth of the primary tumor when it was injected with Salmonella or PBS (upper graphs). *, p<0.05 when comparing the growth of the distal tumor when the primary tumor was either B 16 control or Cx43 shRNAB 16, both injected with Salmonella (lower graph). One of three independent experiments is shown.

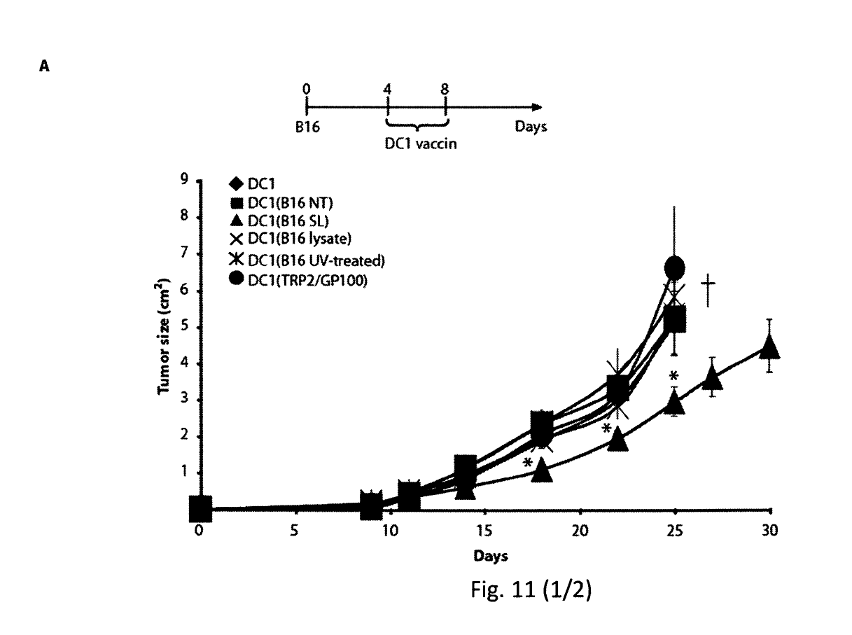

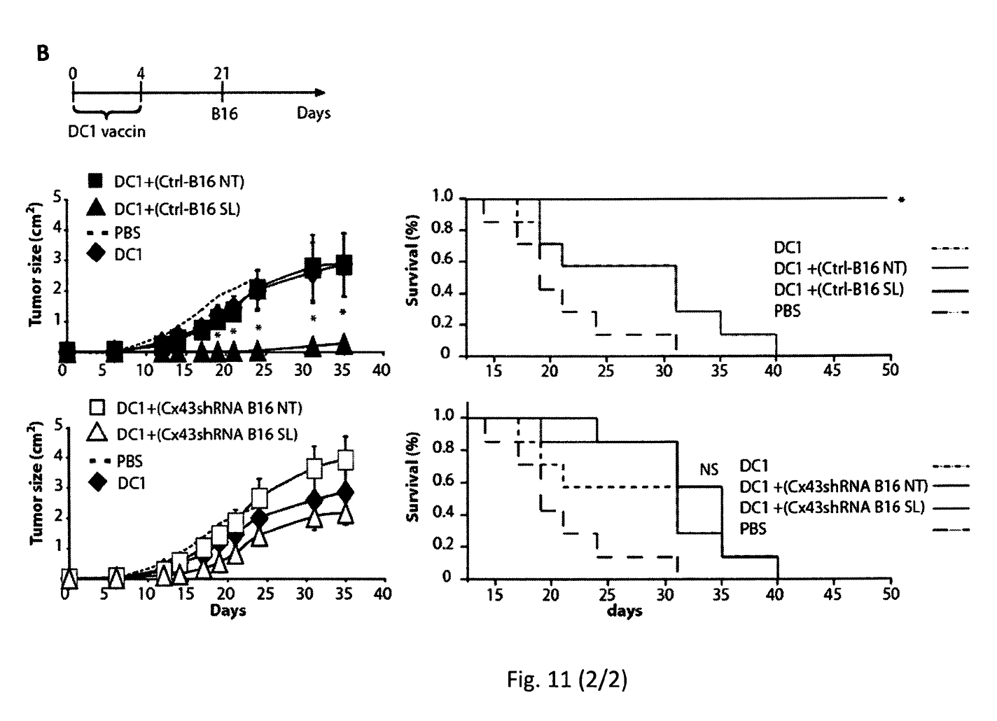

FIG. 11. (A) Gap junction-based vaccination is more effective to induce retardation of the tumor than the current methods of vaccination. C57BL/6J mice were injected with B16 cells and 4 and 8 days later, were vaccinated with DCs loaded as described below. t indicates mice dead before the end of the experiment. Unloaded DCs (DC1, black diamonds), DCs incubated with Salmonella-treated (B 16 SL, black triangles) or not (B16 NT, black squares) B16 cells; DCs loaded with B16 lysate (B16 lysate, black Xs), B16 UV-treated cells (B16 UV-treated, black asterisks) or with a mix of TRP2 and GP100 peptide (TRP2/GP 100, black circles).

(B) Loading of DCs with bacteria-treated tumor cells results in efficient anti-tumor vaccination in a preventive setting. At days 0 and 4 mice (n=8 per group) were vaccinated with DCs loaded as described below, or injected with PBS as control. 21 days later, mice were injected with B16 cells.

Upper graphs: PBS (dashed line), unloaded DCs (DC1, black diamonds), DCs incubated with Salmonella-treated (Ctrl-B 16 SL, black triangles) or not (Ctrl-B 16 NT, black squares) control B16 cells.

Lower graphs: PBS (dashed line), DCs loaded with bacteria-treated (DC1+Cx43shRNAB16 SL, empty triangles) or untreated (DC1+Cx43shRNAB16 NT, empty squares) Cx43shRNAB16.

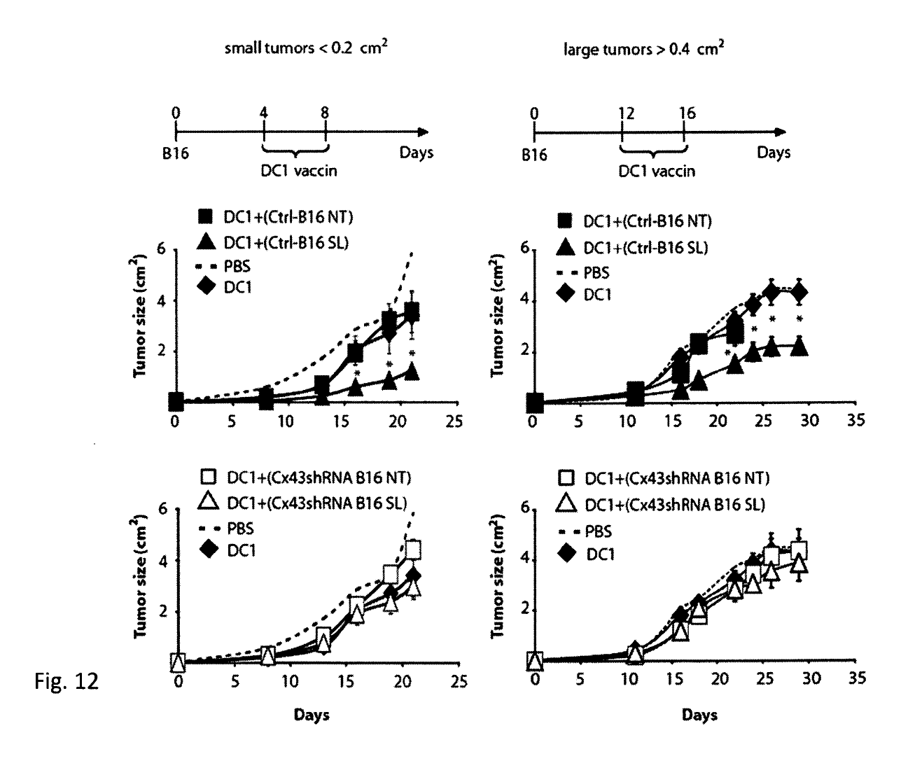

Error bars, s.e. *, p<0.05 when comparing the growth of the tumors of mice vaccinated with DC1+Ctrl-B16 SL versus DC1+Ctrl-B16 NT. NS, not significant. FIG. 12. Loading of DCs with bacteria-treated tumor cells results in efficient anti-tumor vaccination in a therapeutic setting. DCs were incubated with bacteria-treated or untreated B16 cells interfered or not for Cx43. Mice (n=8 per group) were injected with B16 cells and 4 and 8 days later, when the tumor was <0.2 cm² (left graphs), or 12 and 16 days later when the tumor was >0.4 cm² (right graph), mice were vaccinated with DCs purified after loading with tumor cells. The growth of the tumors that did receive PBS as a control (PBS, dashed lines), unloaded DCs (DC1, black diamonds), DCs loaded with bacteria-treated (DC1 +B16 SL, black triangles) or untreated (DC1+B16 NT, black squares) B16, or with bacteria-treated (DC1+Cx43shRNAB16 SL, empty triangles) or untreated (DC1+Cx43shRNAB16 NT, empty squares) Cx43-knocked-down B16 is shown. Error bars, s.e. *, p<0.05 when comparing the growth of the tumors of mice vaccinated with DC1+B16 SL versus DC1+B16 NT.

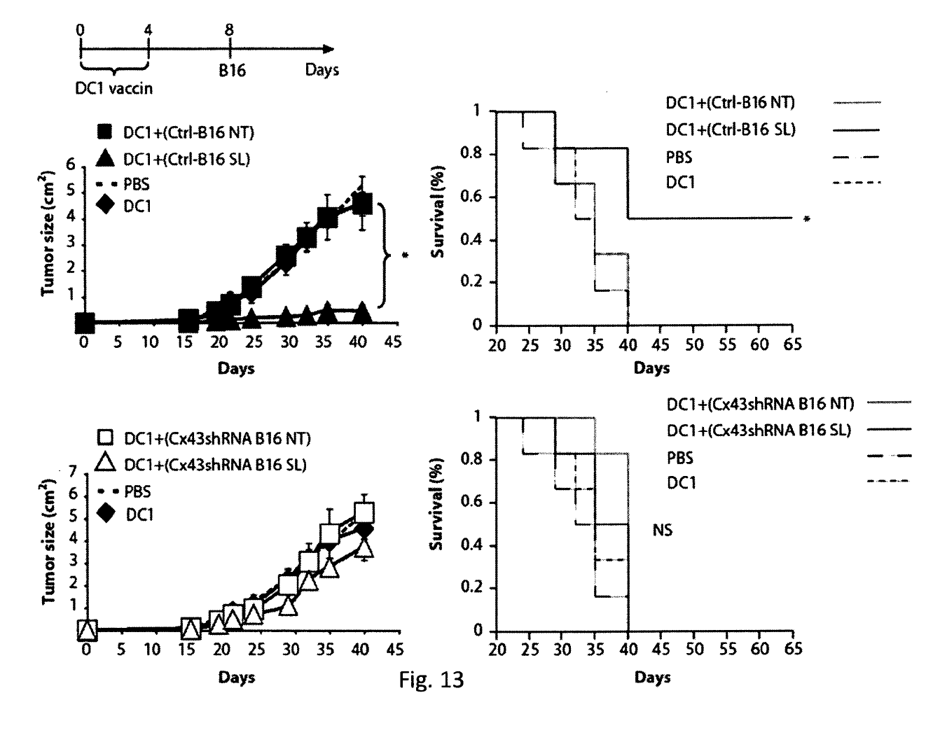

FIG. 13. Loading of DCs with bacteria-treated tumor cells results in efficient anti-tumor vaccination in a preventive setting. At days 0 and 4 mice (n=8 per group) were vaccinated with DCs loaded as described below, or injected with PBS as control. 8 days later, mice were injected with B16 cells. Dashed lines represent the growth of PBS treated tumor-bearing mice. Upper graphs: unloaded DCs (DC1, black diamonds), DCs incubated with Salmonella-treated (B16 SL, black triangles) or not (B16 NT, black squares) B16 cells. Lower graphs: DCs loaded with bacteria-treated (DC1+Cx43shRNAB16 SL, empty triangles) or untreated (DC1+Cx43shRNAB16 NT, empty squares) Cx43shRNAB16. Error bars, s.e. *, p<0.05 when comparing the growth of the tumors of mice vaccinated with DC1+B16 SL versus DC1+B16 NT. NS, not significant.

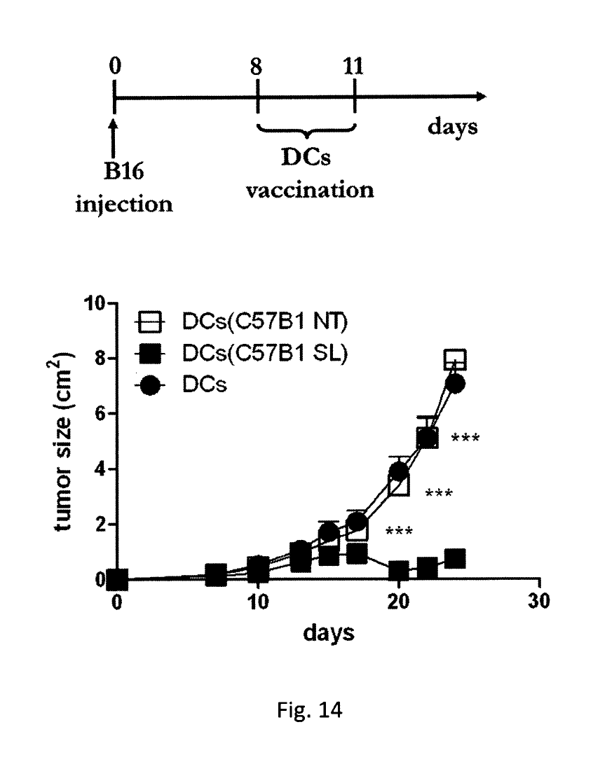

FIG. 14. Cross-protection between different melanomas: DCs cultured with a different SL-infected melanoma are able to protect mice against B16. Mice (n=6 per group) were injected with B16 cells and 8 and 11 days later, mice were vaccinated with DCs incubated with bacteria-treated or untreated C57/B1 melanoma cells, after purification.

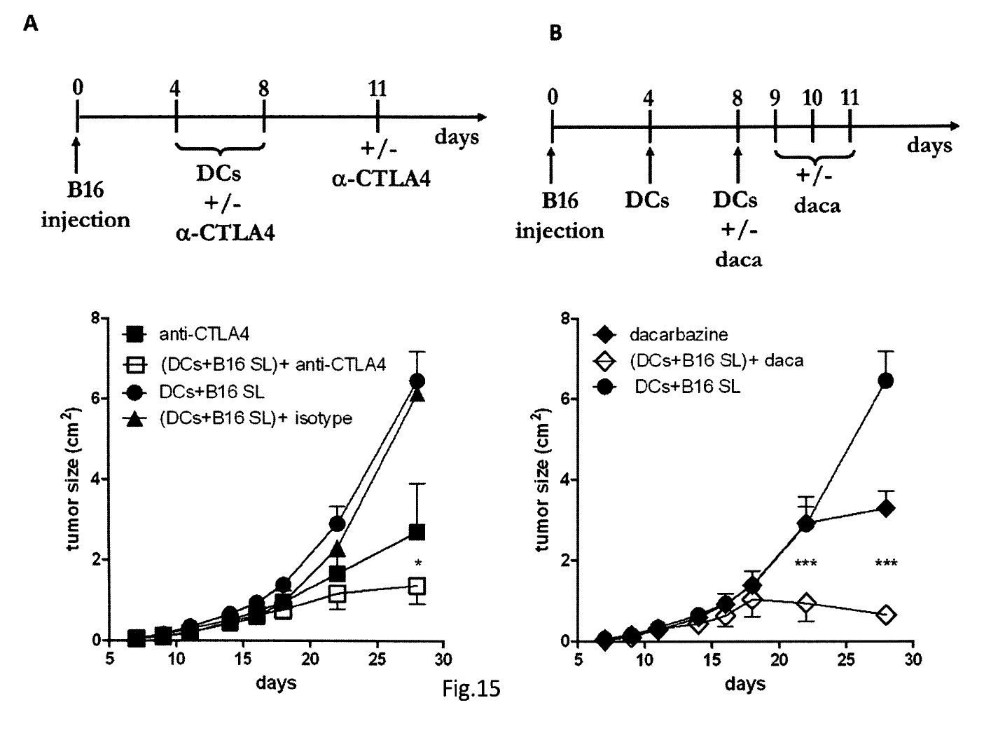

FIG. 15. Combination of vaccination with anti-CTLA4 (A) or dacarbazine (B). DCs were incubated with bacteria-treated or untreated B16 cells. Mice (n=7 per group) were injected with B16 cells and 4 and 8 days later, were vaccinated with DCs purified after loading with tumor cells. Anti-CTLA4 or the isotype control was given intraperitoneally in the same days of the vaccine and at day 11 (100, 50 and 50 μg), while dacarbazine was administered intraperitoneally on day 8 for 4 days (5 mg/kg/day).

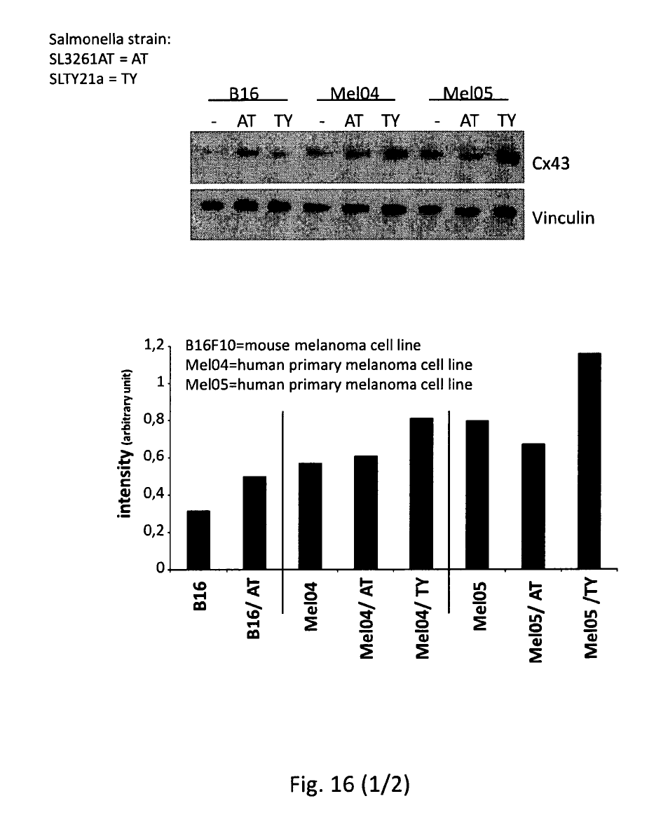

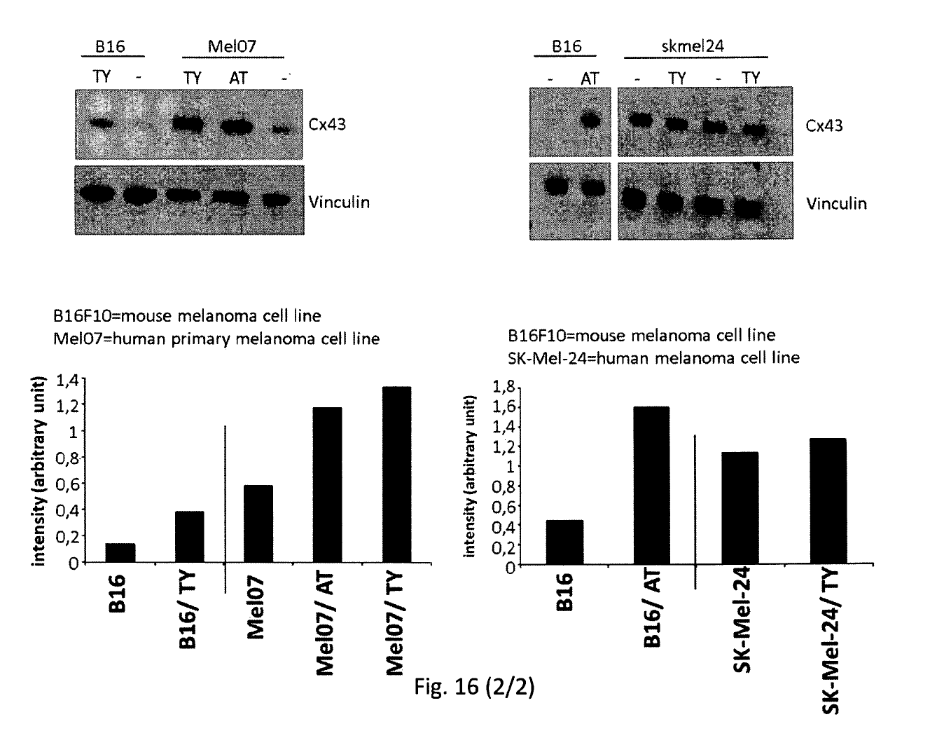

FIG. 16. Salmonella infection induces up-regulation of Cx43 in primary melanoma and melanoma cell lines. Upregulation of Cx43 was evaluated in melanoma cells after infection with different strains of Salmonella (S. typhimurium SL3261AT=AT and S. typhi SLTY21a=TY). Western blot after 24 h using anti-Cx43 or anti-vinculin antibody is shown. Graph bars show band quantification, normalized versus vinculin.

Data are relative to different melanoma cells: B16F10=mouse melanoma cell line, Mel04=human primary melanoma cell line, Mel05=human primary melanoma cell line, Mel07=human primary melanoma cell line and SK-Mel-04=human melanoma cell line.

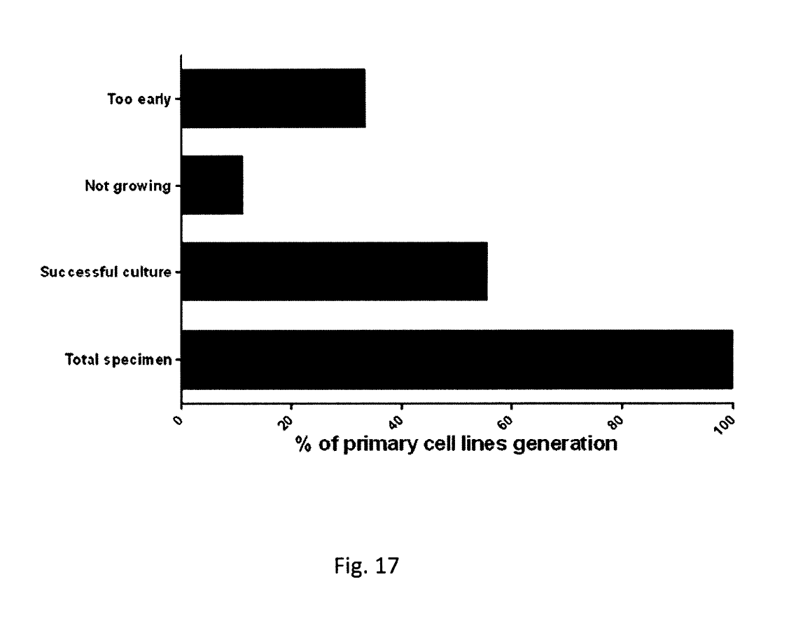

FIG. 17. Cell lines generated from human metastatic melanoma specimens. Fresh melanoma specimens were processed to obtain a single cell suspension.

Tissue specimens were cleaned, cut in small pieces and digested in complete RPMI+collagenase and DNAse at 37° C. for 20 min.

Cells were washed, counted and plated in complete RPMI medium. Initial successful culture was defined by the growth of cells up to passage 4.

Data represent % of successful primary cell line generation from n=9 specimens received.

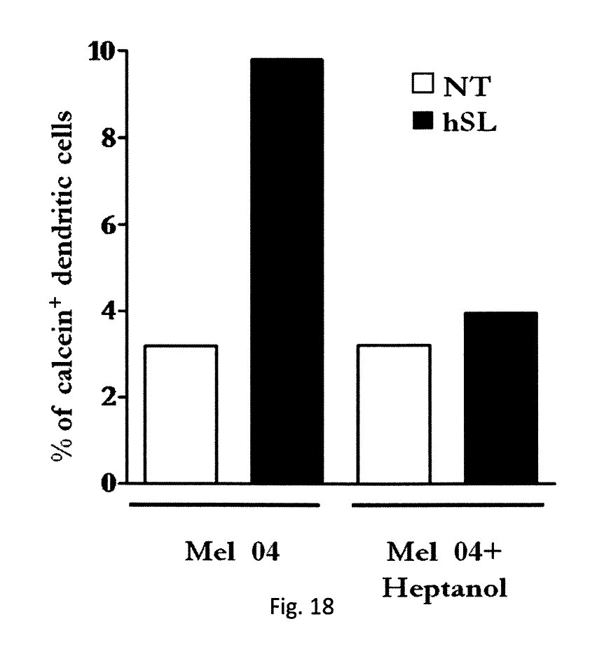

FIG. 18. Salmonella infection upregulates GJs dependent intercellular communication in a melanoma-MoDC coculture model system. Human monocyte-derived DCs (MoDCs) were labeled with DDAO and then extensively washed. Melanoma established or primary cell lines were infected or not with Salmonella and after 24 hours were labeled with calcein-AM. Cells were co-cultured with DCs at a ratio of 1:1 for the indicated time points.

Calcein transfer between tumor cells and DCs was evaluated by cytofluorimetry.

To assess the GJ (Cx43) dependent dye transfer the co-culture of Mel/DCs was carried out in the presence or absence of Heptanol.

It is shown the dye transfer from primary human melanoma Mel-04 to human MoDC. Data represent MoDC that have acquired calcein from melanoma cells. The experiment was performed in the presence or absence of Heptanol, an inhibitor of GJ dependent intercellular communication. Data are expressed as percentage of MoDC—calcein positive cells.

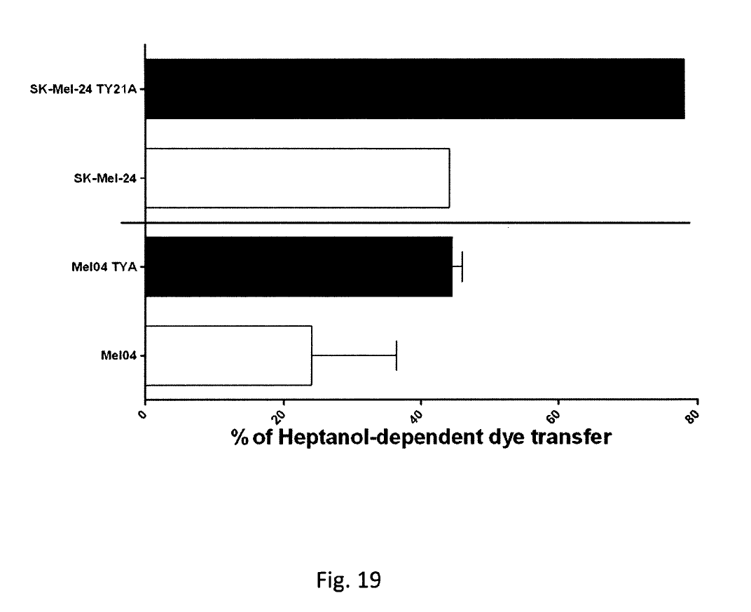

FIG. 19. Salmonella infection increases GJs-dependent intercellular communication from melanoma to dendritic cells.

The dye transfer is performed, as in the previous FIG. 18, from a primary melanoma cells (Mel-04) or a melanoma cell line (SK-Mel-24) to Mo-DC.

Data represent only the dye transfer portion dependent on GJ (Heptanol dependent). Data are expressed as percentage of heptanol dependent dye transfer.

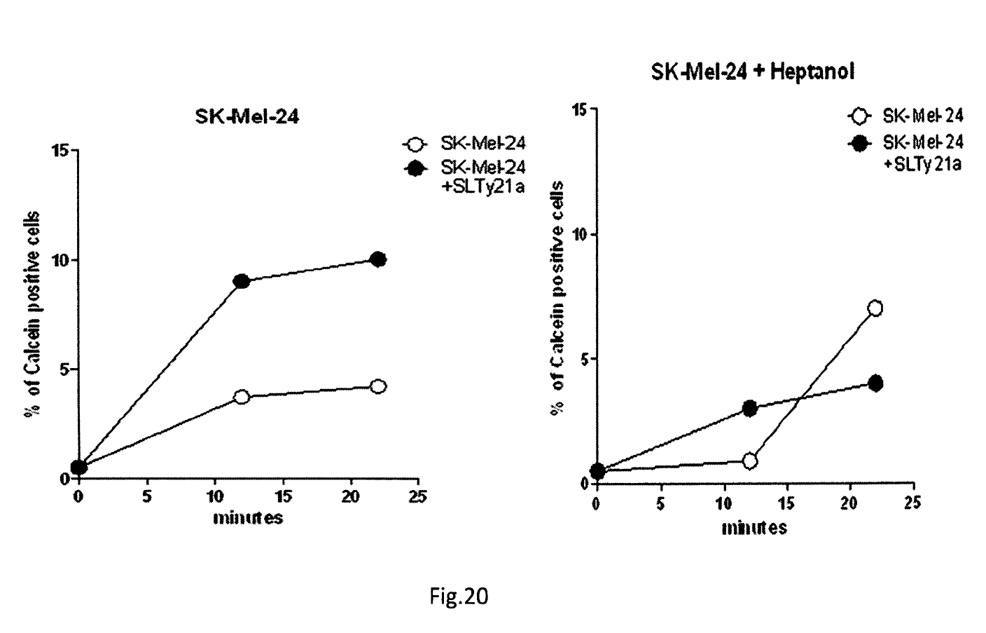

FIG. 20. Salmonella infection up-regulates GJs dependent intercellular communication in SK-Mel-24 melanoma cell line. In this experiment the dye transfer was performed from melanoma to melanoma cells (SK-Mel-24). The increase in dye transfer from Salmonella infected to non-infected SK-Mel-24 cells is clear as compared to that from non-infected to non-infected cells. The right panel represents the same experiment performed in the presence of Heptanol, an inhibitor of GJ dependent intercellular communication. Data are expressed as percentage of SK-Mel-24 calcein positive cells.

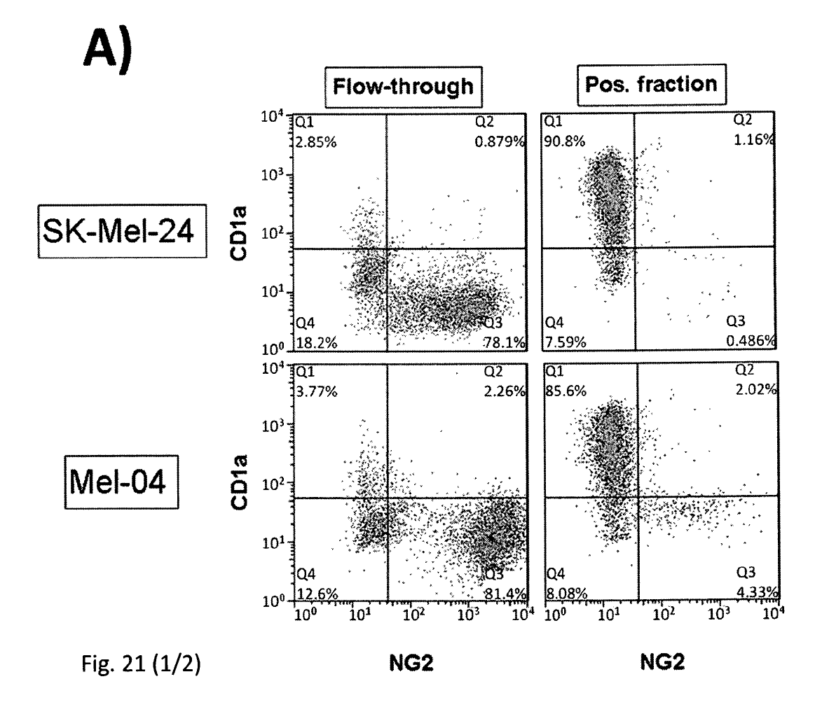

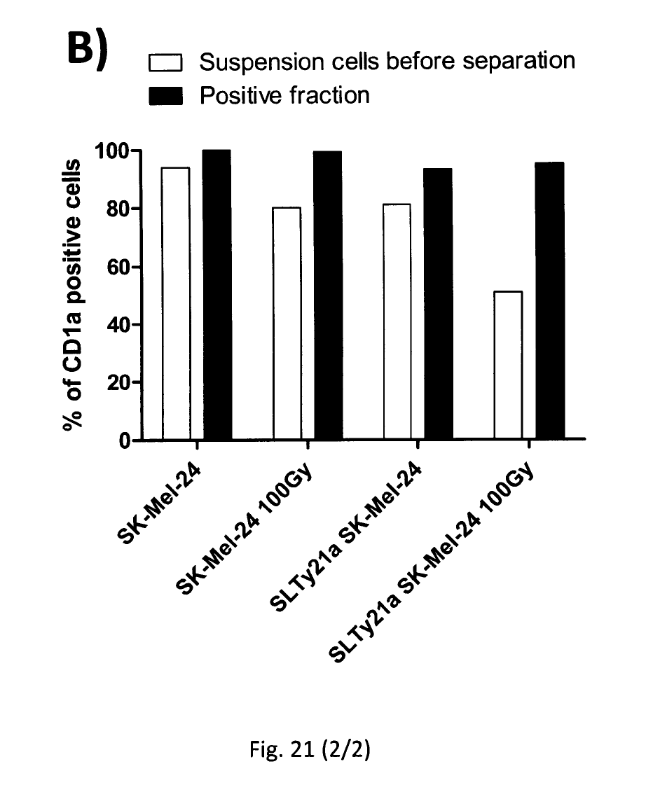

FIG. 21. Dendritic cell purification from melanoma coculture by CD1c positive selection, a GMP available, monoclonal antibody. MoDC were cocultured either with melanoma primary cell line (mel04) or SK-Mel-24 cells. Cells in suspension, containing MoDC and residual melanoma cells, were harvested and underwent a purification step by magnetic cell sorting. Briefly, cell suspension was labeled with CD1c biotinilated mAb followed by anti-biotin microbeads (Miltenyi Biotech). Labeled cells were loaded onto a magnetic column allowing only the retention of cells labeled by CD1c mAb (MoDC), the flow-through contained mainly melanoma cells (CD1c negative).

Panel A) dot plots from cytofluorimetry analysis representing CD1a (MoDC) and NG2 (melanoma cells) are shown.

Panel B) MoDC were cocultured with SK-Mel-24 infected or not with SL-TY21a and irradiated at 100 Gy. Percentage of MoDC present in the suspension (total) and column retained CD1c cell (positive) is shown.

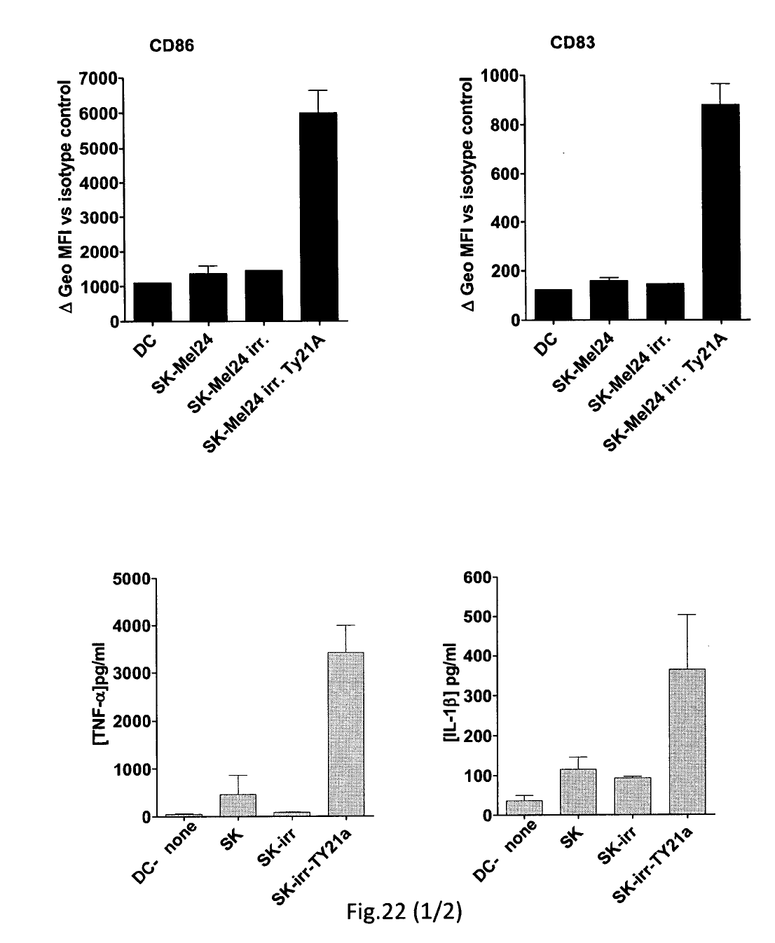

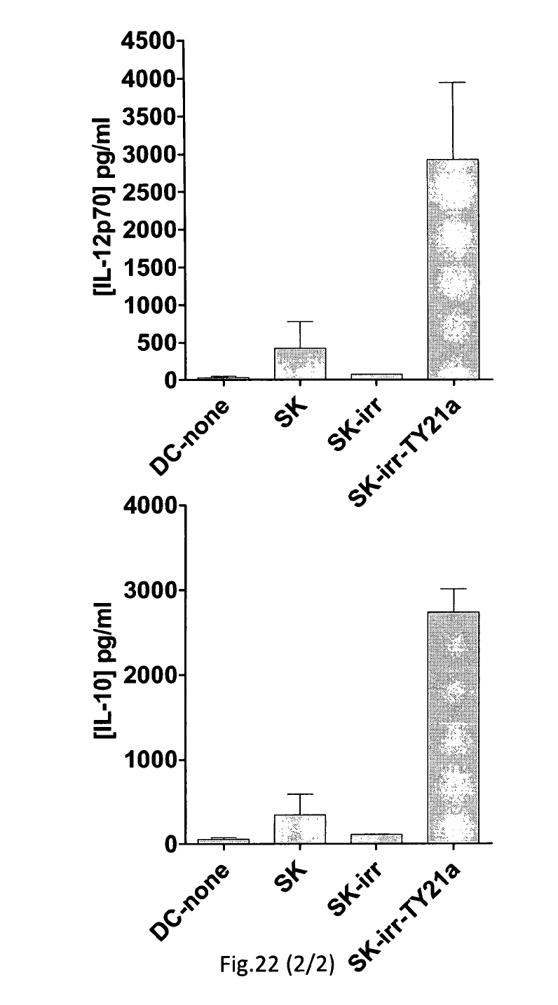

FIG. 22. Immature Mo-DCs up-regulate co-activatory markers and cytokines after coculture with SL-infected melanoma cells. MoDC were cocultured with Mel-04 (primary melanoma cells), and after 24 h floating cells containing MoDC, were harvested. Cells were stained with an anti-CD86 and CD83 in order to evaluate maturation/activation marker expression. Culture supernatants were analyzed for cytokine secretion (IL-12p70, IL-1b, TNF-a and IL-10) by BD Cytometric Bead Array (CBA). Bar graphs represent the percentage or the amount of the indicated molecules measured.

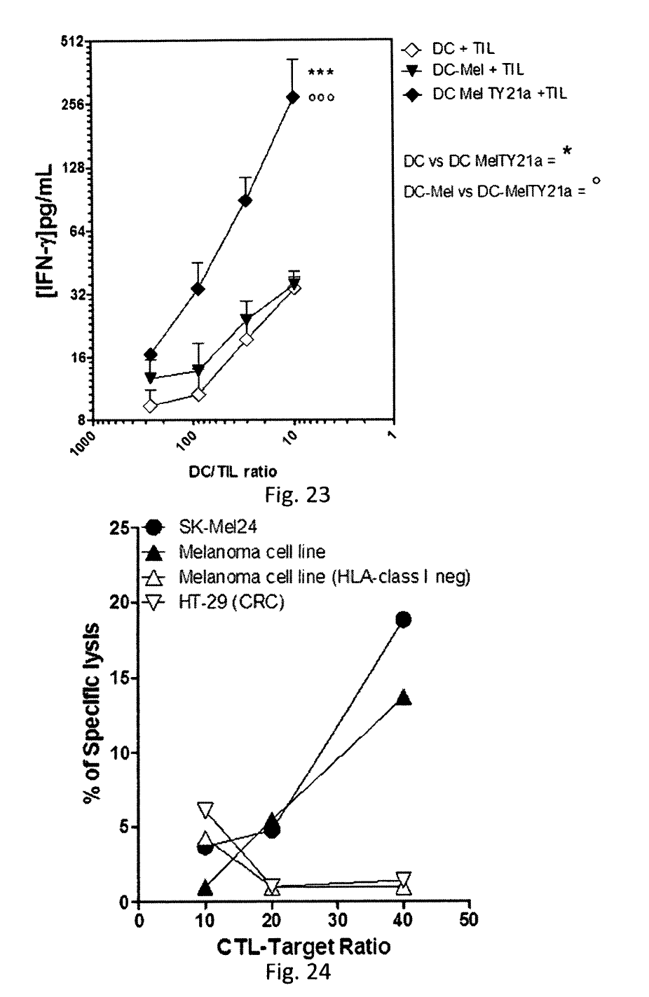

FIG. 23. Salmonella-infected melanoma cell lines dictate the capacity of autologous Mo-DC to activate metastatic melanoma-derived Tumor Infiltrate Lymphocyte.

TIL were cultured at 1:10 ratio with autologous MoDCs, previously loaded with autologous infected and non-infected primary melanoma cells, for 5 days.

Data represent amount of IFN-γ level in the culture supernatant.

FIG. 24. SL-infected SK-Mel-24 loaded MoDC generate CTLs able to recognize epitopes shared among melanoma cells. CTL generated as described in materials and methods were tested for their antigen-specific cytotoxicity using DELFIA cytotoxicity kit. Target cells were: SK-Mel24, melanoma cell line, HLA negative cell line, HT29 (colon carcinoma).

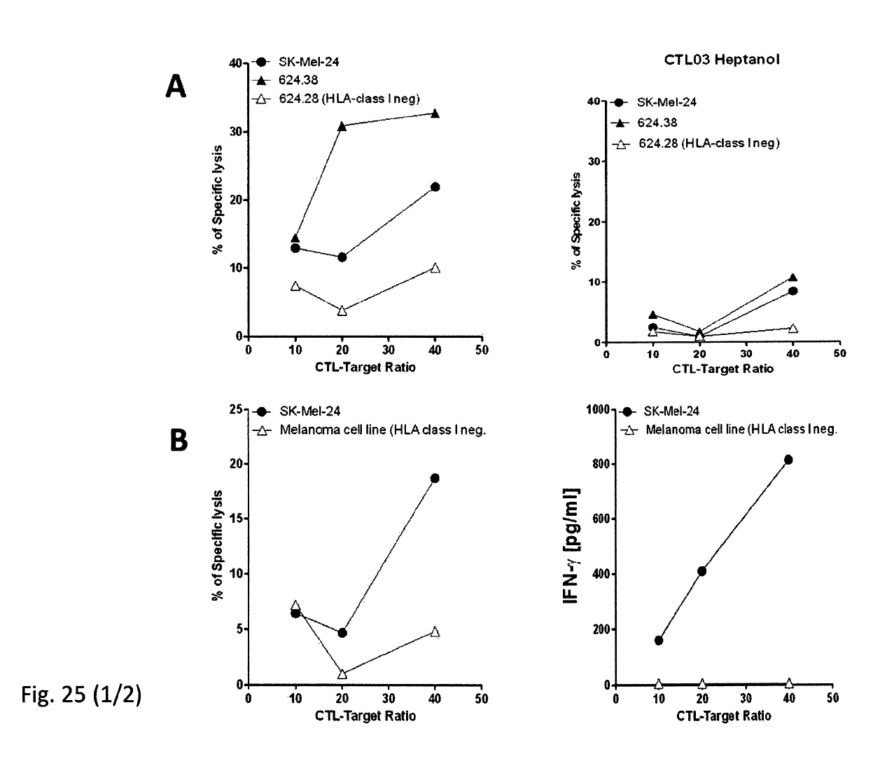

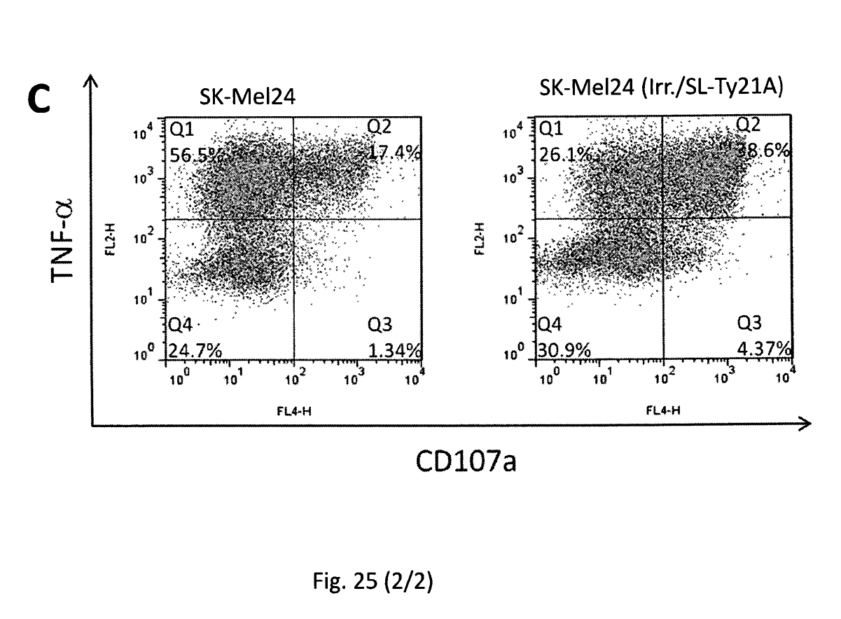

FIG. 25. Gap-junction dependent interaction through MoDC and SL-infected SK-Mel-24 is necessary to generate CTL response. A, percentage of specific cytotoxicity of CTLs generated with SL-infected SK-Mel-24 loaded MoDCs supplemented during the coculture with (right) or without (left) heptanol. Functional activation of CTL. B, Cytotoxic activity of melanoma specific CTLs (left) is paralleled by IFN-g production (right) in culture supernatant. C, Dot plot analysis of TNF-a versus CD107a is shown.

### Results

Bacteria induce the upregulation of Cx43 in several tumor cell lines.

The first requirement for the establishment of GJ between cells is the expression of Connexins. Cx43 has been shown to be ubiquitous and to be implicated in immune responses (7) but also to be downregulated during melanoma progression (25). Indeed, GJIC is lost in many tumors enabling the autonomous cell behavior of transformed cells (9). In this study authors used the highly aggressive and low immunogenic B16F10 melanoma model (named B16 from hereafter). Hence, authors first tested whether B16 cells expressed the ubiquitous Cx43. While untreated B16 cells only faintly expressed Cx43, it was upregulated after infection with Salmonella as assessed by immunofluorescence and Western blot analysis at 24 and 48 h (FIG. 1A-B). The same upregulation of Cx43 was observed when B16 cells were incubated with purified bacterial components, like lypopolysaccharide (LPS), lipoteichoic acid (LTA) and flagellin (FIG. 1C). A dendritic cell line called DC1 treated with LPS was used as positive control for Cx43 upregulation (FIG. 1B). Because it has been shown that IFN-gcould also upregulate the expression of Cx43 in immune cells (12), authors tested whether IFN-g could induce the expression of Cx43 in B16 cells. Authors found that not only IFN-g upregulated Cx43, but it also acted in synergy with the bacterial components (FIG. 1C). Authors then analyzed whether Cx43 was upregulated also in tumors infected with Salmonella in vivo. Mice were injected subcutaneously with 10⁵ B16 cells in the right flank. After 10 days, when the tumor reached a size of 0.5 cm², tumors were infected with an avirulent strain of Salmonella typhimurium (SL3261AT). 1 and 3 days later, tumors were resected and Cx43 expression was analyzed by FACS analysis. As shown in FIG. 1D, Salmonella treatment induced the upregulation of Cx43 also in vivo. Authors then analyzed whether IFN-gexpression was induced in vivo after intratumoral bacterial infection. As shown in FIG. 1E, IFN-gwas detected in infected tumors already one day after infection reaching a peak at 7 days (FIG. 1E). This suggests that there may be a synergy in vivo for the upregulation of Cx43 in infected cells, but also non-infected cells could express Cx43 in the inflamed environment via the action of free bacterial components or IFN-g. To evaluate whether this was a peculiar characteristic of the tumor cell line that authors used, authors extended the analysis to two additional murine (B16BL6 and C57B1) and to several human melanoma cell lines (WM-115, WM-266.4, SK-MEL-31, CHL-1, IGR-1, IGR-37, IGR-39, MEWO, RPMI-7951). All tested murine tumors and half of human cell lines had a behavior similar to that of B16 in response to bacteria, whereas the other half of human melanoma cell lines expressed higher basal level of Cx43, which was either unchanged or downregulated in response to bacteria (FIG. 2). In addition, the expression of Cx43 was also tested in other types of human tumors, and authors found that Salmonella is able to upregulate Cx43 also in the lung carcinoma A549, in the colorectal adenocarcinoma Caco-2, and in the prostate adenocarcinoma PC-3 (FIG. 3A). Worthy of note, Salmonella is also able to upregulate Cx43 in non solid tumors like the leukemia NB4 and the T cell-lymphoma Karpas-299 (FIG. 3B).

Thus, whereas tumor cells are generally induced to lose Cx43 expression, treatment with bacteria and/or IFN-g resulted in Cx43 upregulation in all tested murine and in those human cell lines that maintained Cx43 in a downregulated state also in culture.

Salmonella modulate the expression of other Connexins in both mouse and human melanoma cells.

Although Cx43 is ubiquitously expressed, authors wanted to assess whether Salmonella could modulate the expression of other Connexins. mRNA expression of connexins 45, 46, 47 in B16 cells and connexins 40, 45, 46, 47, and 50 in human melanoma cell lines IGR37, IGR39 and WM266.4 were analyzed in response to Salmonella in combination with IFN-g (SL+IFN) or not (NT). As shown in FIG. 4, also connexins 45, 47 in B16 cells (a) and connexins 40, 47, and 50 in human melanoma cell lines (b) were upregulated in response to bacterial stimulation, indicating a broad control of connexin expression by Salmonella.

Cx43 upregulation correlates with the generation of functional gap junctions.

Having shown that Cx43 was similarly regulated in other tumor cell lines, authors tested whether Cx43 upregulation correlated with the formation of functional GJ pores. Infected or non-infected B16 cells were microinjected with a mixture of a GJ-diffusible dye (Lucifer yellow, LY) and GJ-nondiffusible dye (Dextran Texas Red, 70 kDa) (13). Only after bacterial infection the LY was able to diffuse to adjacent cells (FIG. 5A), at a level comparable to that of untreated NIH-3T3 cells used as a positive control (26). Authors then addressed whether a similar intercellular communication was established also with adjacent DCs. 24 h after the infection, infected and not-infected B16 tumor cells were labeled with the GJ-diffusible dye calcein-AM and LPS-treated DCs were labeled with the non-transferable dye 7-hydroxy-9H(I,3-dichloro-9,9-dimethylacridin-2-one) (DDAO). The cells were plated separately but in close proximity in the same Petri dish and the transfer of dye from the B16 cells to DCs was monitored by real time confocal videomicroscopy. As shown in the sequence of collected frames (FIG. 5B), starting from 20 min, the transfer of the calcein dye was observed from tumor cells (white) to DCs (red), which gradually became yellow. Interestingly, also the DCs were able to transfer the dye among themselves indicating that GJIC was occurring also among the DCs. In order to quantify the transfer of material occurring between tumor cells and DCs authors used a more quantitative cytofluorimetric method to measure GJIC using the same dyes to distinguish donor from acceptor cells. After one hour of co-culture, the number of DDAO⁺Calcein-AM⁺ cells was significantly increased when tumor cells were pretreated with bacteria (FIG. 5C). This assay also allowed us to evaluate whether the transfer of the dye was indeed occurring via GJs because the GJ uncoupler heptanol, which is a pleiotropic lypophilic agent that blocks electrical cell-to-cell communication, completely abolished the transfer of calcein from B16 cells to DCs (FIG. 5C). This indicates that functional GJs can be formed between tumor cells and DCs for cell-cell communication and this mechanism may be used for the transfer of antigenic material.

Dendritic cells present preprocessed tumor-derived peptide without the need of phagocytosis.

To follow the transfer of antigenic material from infected tumor cells to DCs, authors used a B 16 cell line expressing the model antigen Ovalbumin (B16-OVA). Authors analyzed the appearance of the OVA₂₅₇₋₂₆₄ SIINFEKL peptide in association with MHC class I (K^{b}) molecules using the specific antibody 25-D1.16 (27) on the surface of DCs

To assess whether DCs acquired the peptide after phagocytosis of infected B16-OVA cells authors labeled the latter with the vital dye CFSE and incubated stained cells with DCs for 24 h. Authors detected DCs positive for 25-D1.16 antibody primarily in the negative fraction of CFSE stained cells (CD11c⁺ CFSE⁻ cells) and only after infection of tumor cells (FIG. 6A). The low percentage of k^{b}OVA positive cells in the CFSE⁺ DC fraction after incubation with B16 control is likely due to an unspecific binding of the antibody. This suggests that phagocytosis of intact tumor cells or apoptotic bodies is not required for the exchange of the tumor-associated peptide from infected tumor cells to DCs. To address the nature of the transfer, heptanol was added to the cell culture. Heptanol completely abolished the presentation of the peptide, indicating that the OVA peptide is likely transferred via GJIC (FIG. 6A).

Since components of bacteria are able to induce Cx43 upregulation in DCs (12), authors assess whether the infection of the DCs-tumor cells coculture could improve the transfer of OVA peptide. Thus, authors treated with Salmonella the tumor cells alone (B16 and B16-OVA) or the coculture DCs-tumor cells, in presence or absence of Heptanol. After 24 h of coculture authors analyzed the presentation of OVA peptide in complex with MHC class I. The comparison between the two methods shows that the presentation of OVA peptide in complex with MHC class I is even better in case of the infection of the coculture (FIG. 7).

Because only linear peptides up to 16 amino acids can be transferred via GJ, the exploitation of this pathway requires processing of the antigenic material within tumor cells. Thus, authors examined whether proteasome dependent degradation in tumor cells was necessary for the presentation of the OVA peptide by DCs. Pretreatment of B16-OVA cells with lactacystin, a cell permeable irreversible 20/26S proteasome inhibitor (28), prevented the DC surface exposure of the K^{b}-OVA complex (FIG. 6B).

Authors then evaluated whether the presentation of peptides via GJ led to T cell activation and whether this mechanism was dependent on Cx43 expression by tumor cells. Authors generated a clone of B16-OVA cells stably silenced for the expression of Cx43. Authors used four different lentivirus constructs but only three were effective in silencing Cx43 (not shown), hence we used the non-effective one as a negative control. In FIG. 8A, the extent of Cx43 silencing is shown. Authors then tested T cell activation in terms of IFN-grelease using purified naive OVA₂₅₇₋₂₆₄-(SIINFEKL)-specific OTI T cells. Authors confirmed that pretreatment of B16-OVA with Salmonella favored the cross-presentation of OVA peptide by DCs (FIG. 8B). This presentation was partly dependent on Cx43 as its silencing in the B16-OVA strongly reduced the capacity of DCs to activate OTI T cells (FIG. 8B). The residual activation of T cells may be due to alternative cross-presentation pathways, or to incomplete silencing of Cx43 in the B16-OVA cells. Together, these data suggest that bacterial infection promotes the transfer of a processed peptide via a GJ-dependent mechanism.

Intratumoral bacterial injection increases the percentage of CX43⁺ DCs in lymph nodes.

As shown in FIG. 5B, the transfer of GJ-diffusible dye occurs not only between tumor cells and DCs, but also among DCs. This could be a powerful mean to increase the number of DCs capable to present tumor-associated antigens in the draining lymph nodes. Hence, authors tested the frequency of CD11c⁺Cx43⁺DCs in lymph nodes draining or not the tumor site that was treated with Salmonella or PBS, as a control. Authors observed that already 24 h after infection the frequency of CD11c⁺Cx43⁺ CD4⁺ DCs was increased in lymph nodes both draining and non draining the tumor site, but only if the tumor site (or the skin) was infected with Salmonella (FIG. 9A). At later time points (3 days), also the CD8⁺ subset of DCs expressing Cx43 was elevated in frequency (FIG. 9A). This coincided with an increase of activated DCs as shown by the upregulation of the activation marker CD86 (FIG. 9B). Activated DCs were actually the ones expressing Cx43 (FIG. 9C). Authors were quite puzzled to observe an increase of Cx43⁺ DCs also in non-draining lymph nodes as authors previously showed that Salmonella remains confined to the infected site, via the generation of a granuloma-like structure (29, 30). However, authors found an increased concentration of IFN-gin the serum of mice whose tumors were infected (FIG. 9D).

Interestingly authors could not find IFN-gin the serum of Salmonella-infected mice not bearing tumors. This may be due to the nature of the melanoma that is highly vascularised.

Having shown that IFN-gcan autonomously induce the upregulation of Cx43, this may explain the increase in Cx43⁺ DCs in non-draining lymph nodes suggesting a systemic inflammatory effect of the Salmonella, as authors recently described (24).

Cx43-dependent cross-presentation is the major mechanism of tumor-antigen cross-presentation in vivo.

In our previous studies authors observed that intratumoral injection of Salmonella led to increased cross-presentation of tumor antigens by DCs and to the induction of a systemic anti-tumor response that was effective in retarding the growth of distant, untreated masses (23, 24). As the intratumoral injection of Salmonella leads to a local inflammatory response and necrosis of tumor cells, many mechanisms of cross presentation (31), including the capture of dying cells (32) or of released soluble proteins (33), could account for tumor antigen cross-presentation. Hence, authors analyzed the contribution of a GJ-dependent cross-presentation pathway in vivo after bacterial infection. Authors hypothesized that if Cx43 was required to initiate a systemic anti-tumor response via GJs, its absence in the Salmonella-treated tumor would impact on the growth of a distant Cx43-WT untreated tumor. Authors generated a cell line of B16 cells stably silenced for Cx43 that conserved gene silencing till the end of the experiment (FIG. 10A). Authors used as a control a B16 cell line transfected with a lentivirus carrying an shRNA ineffective in silencing Cx43 (Ctrl-B16). B16 control (Ctrl-B16) or B16Cx43shRNA cells were inoculated in the right flank, while 3 days later B16-WT cells were inoculated in the left flank. At days 11 and 15 from the first tumor challenge, Salmonella or PBS as a control, was injected only in the right tumor. The growth of the two tumors was followed over time. The tumors treated with bacteria regressed regardless of the presence or absence of Cx43 (FIG. 10B). This is not surprising as the initial effect of killing of tumor-infected cells is independent on the activation of an adaptive immune response to the tumor (23). The growth of the distal untreated tumors instead was controlled only when the tumors injected with bacteria expressed Cx43 (FIG. 10B). In contrast, authors found that the antitumor response on the distal tumor was abrogated when the Salmonella treated tumor was silenced for Cx43 (FIG. 10B). This suggests not only that Cx43 is the principal component of GJ in vivo but also that this pathway plays a prominent role in Salmonella-induced tumor antigen cross-presentation. To confirm that the effect on the distant untreated tumor was mediated by the induction of a cytotoxic T cell response generated by DCs receiving the tumor peptides from infected cells, authors deleted cytotoxic T cells using a neutralizing antibody to CD8 during the course of the experiment, following the schedule reported in FIG. 10B. An isotype IgG control antibody was used as a control of the experiment. As expected, neutralization of CD8⁺ T cells abolished the protective response on the distal untreated tumor (FIG. 10B right panel). These experiments indicate that in vivo the Cx43-dependent mechanism of cross-presentation of tumor antigens in response to bacterial infection is dominant over other cross-presentation pathways and is required to generate an effective anti-tumor response.

DCs loaded in vitro with bacteria-treated tumor cells are potent in inducing anti-tumor immunity.

For many years DCs have been proposed as nature's adjuvants in cancer immunotherapy (34). However, although DCs have proven to be very good at inducing a strong immunological response in treated patients, the clinical response in these patients has been very modest. Among the several reasons for this failure, one possible explanation may be that as DCs have a different proteasome than tumor cells, they may generate peptides that are not readily produced within tumor cells. Hence, tumor cells do not present the peptides to which cytotoxic T cells are specific. A GJ-dependent mechanism of cross-presentation that instead is based on pre-processing of antigenic peptides within tumor cells may overcome this drawback. Thus, authors compared the effectiveness of a GJ-dependent pathway of MHC class I loading with those generally used in the clinics (peptides, tumor lysates and apoptotic bodies). Authors incubated DCs with either purified peptides (GP100 and Trp-2), tumor cell lysates obtained with freezing and thawing of B16 tumor cells, or with UV-irradiated B16 cells. Alternatively, authors loaded DCs with infected or non-infected B16 cells silenced or not for Cx43. Authors purified the DCs (>98% purity) by positive selection and injected them s.c. after Mitomycin-C treatment to avoid the growth of any possible contaminant tumor cells in mice challenged 4-days earlier with B16 cells (in a therapeutic setting). Authors found that the only pathway leading to an effective anti-tumor response was the one employing DCs loaded with infected B16 cells (FIG. 11A). This pathway was effective both on small (<0.2 cm²) and large (>0.4 cm²) tumors and was dependent on Cx43 as the anti-tumor response was lost if the DCs were loaded with a B16 that was silenced for the expression of Cx43 (FIG. 12). As it was shown that transfer of peptides between DCs could also contribute to cross-presentation (35), authors decided to silence the expression of Cx43 only in tumor cells to avoid to inhibit DC-DC peptide transfer.

Even more striking was the effect observed in a preventive setting. Indeed if authors vaccinated the mice with bacteria-treated B16 cells twice (at day 0 and 4) before the challenge with B16 cells (at day 21), the tumor growth was strongly inhibited and 100% of the mice remained tumor free and survived 50 days after tumor challenge (FIG. 11B). Again this effect was dependent on Cx43 as it was abolished when using Cx43-silenced B16 cells to load the DCs (FIG. 11B). When the tumor was injected at earlier time points (day 8 after vaccination) tumors grew more slowly in mice receiving DCs loaded with bacteria-treated B16 cells but only 50% of the mice survived (FIG. 13). Note that tumor growth is shown till day 40 when mice carrying 4 cm² tumors were sacrificed.

Together, these results indicate that the transfer of antigenic peptides from tumor cells to DCs via GJs is far more effective than standard pathways of DC-loading to generate protective DC-based vaccines.

Authors then assessed if their protocol could work also in an heterologous setting using a different SL-infected melanoma cell line to “load” DCs against B16 melanoma.

For this purpose authors vaccinated B16-bearing mice, at day 8 and 11 after tumor inoculation, with DCs cultured with the melanoma C57B1 infected or not with Salmonella. As shown in FIG. 14, the vaccination was effective only when SL-infected C57B1 cells were used to “load” DCs, compared to the not treated C57B1 cells. This experiment shows that there are melanoma-shared peptides that can be transferred from a tumor (i.e. C57B1) to DCs and then these DCs are able to vaccinate mice against another tumor (i.e. B16). Finally, in order to improve the efficacy of the therapeutic vaccination authors combined their protocol with the anti-Cytotoxic T Lymphocyte Antigen 4 (CTLA4) treatment, or with the chemotherapeutic drug dacarbazine. In the first case, the antibody binds to CTLA4 thus inhibiting the negative regulation of T cell proliferation, while in the second case dacarbazine is an alkylating agent which kills cancer cells by adding an alkyl group to its DNA. Both combined vaccinations [(DCs+B16 SL)+anti-CTLA4 and (DCs+B16 SL)+daca] have an higher efficacy compared to the vaccination alone (DCs+B16 SL) and to the single treatment with anti-CTLA4 or dacarbazine (FIG. 15).

Results Obtained With Primary Human Cells

Salmonella infection induces up-regulation of CX43 in human primary melanoma and melanoma cell lines.

As shown already in FIG. 2 authors have demonstrated that human melanoma cell lines upregulated the expression of Cx43. So authors evaluated whether also primary melanoma cell lines generated from patients upregulated Cx43 in response to Salmonella. Authors also assessed whether the strain of Salmonella typhi that is currently used for thypoid vaccination (Vivotif: Ty21A) and in a clinical protocol of intratumoral salmonella vaccination that authors are carrying out at their Institute had the same properties as the murine Salmonella typhimurium strain. Upregulation of CX43 was evaluated, in melanoma cells, after infection with either SL3261AT=AT or SLTY21a=TY. Western blot after 24 h using anti-Cx43 or anti-vinculin antibody is shown in FIG. 16. Authors observed an upregulation of Cx43 in three human primary melanoma cell lines (Mel04, Mel05, Mel07) and the vaccine strain Ty21A was even stronger than the murine strain. Ty21A induced the upregulation of Cx43 also in the universal melanoma cell line SK-Mel-24 although to a lesser extent than in the primary human melanoma cell lines.

Cell lines generated from human metastatic melanoma specimens.

It is reported in the literature that it is possible to generate melanoma cell lines from nearly 60% of patients (Dillman, 2009 ibidem). Hence, authors wanted to assess in their hands what was the percentage of cell lines that authors could generate. Fresh melanoma specimens were processed to obtain a single cell suspension and cell lines were generated as reported in the methods section. Initial successful culture was considered as the growth of cells up to passage 4.

In line with what described in the literature authors obtained cell lines from nearly 60% of specimens. Data represent % of successful primary cell line generation from n=9 specimens received (FIG. 17).

Salmonella infection up-regulates GJs dependent intercellular communication in a melanoma-moDC coculture model system.

Authors then assessed whether upregulation of Cx43 by Salmonella correlated with increased functional gap junctions. DCs were labeled with 1 μM of DDAO, while established melanoma or primary cell lines were infected or not with Salmonella and after 24 hours were labeled with 0.5 μM calcein-AM. Cells were co-cultured with DCs at a ratio of 1:1 for the indicated time points.

Calcein transfer between tumor cells and DCs was evaluated by cytofluorimetry. To assess the GJ (CX43) dependent dye transfer the co-culture of Mel/DCs was carried out in the presence or absence of Heptanol that blocks gap junction intercellular communication. FIG. 18, shows that dye transfer can occur also between human Monocyte derived (MO)DCs and human melanoma cells. Graph shows the dye transfer from primary human melanoma Mel-04 to human MoDC. In both cases pre-treatment of tumor cells with Salmonella facilitates dye transfer and this occurs via GJ as heptanol is capable of inhibiting the transfer.

Salmonella infection increases GJs-dependent intercellular communication from SK-Mel-24 to dendritic cells.

Since the generation of a primary cell line from the patient was not efficient and fast, authors explored the possibility to use an “universal” cell line as a source of melanoma antigens. For this purpose authors used the SK-Mel-24, which is already known to express most of the common melanoma associated antigens and it is already used in vitro for the extraction of naturally processed melanoma peptides (Imro MA, et al., Cancer Res. 1999 May 15;59(10):2287-91). Moreover, authors have already shown, in FIG. 14, that it is possible to vaccinate mice against B16 using DCs cultured with another SL-infected melanoma, thus confirming cross-protection between different melanoma cell lines.

Authors then evaluated whether also SK-Mel-24 was induced to establish functional GJ with MoDCs. The dye transfer was performed, as in the previous FIG. 18, from primary human melanoma cells (Mel-04) or the universal melanoma cell line (SK-Mel-24) to Mo-DC. Also in this case, preincubation of tumor cells with Salmonella Ty21A favored the formation of functional GJ as most of the calcein transfer was inhibited in the presence of Heptanol (FIG. 19).

Salmonella infection up-regulates GJs dependent intercellular communication between SK-Mel-24 melanoma cells.

Authors then evaluated whether salmonella infection favored cell to cell communication between infected and non infected melanoma cells (SK-Mel-24). Also in this case authors could clearly observe a Salmonella-induced increase in total dye transfer from infected to non infected SK-Mel-24 cells (FIG. 20 Left panel). Again this transfer was dependent on GJ as it was inhibited in the presence of heptanol (FIG. 20 Right panel).

Dendritic cell purification from melanoma coculture by CD1c positive selection: a GMP available, monoclonal antibody.

In the proposed clinical protocol authors will separate DCs from irradiated melanoma cells before infusion in the patient. Although already the MODC-melanoma coculture cell suspension is enriched in MoDCs that detach from the cell culture dish, authors wanted to analyze whether authors could purify DCs to more than 90%. In order to identify a possible, GMP available, monoclonal antibody to purify MoDCs from melanoma-MoDC coculture, cells were cocultured either with a melanoma primary cell line (mel04) or SK-Mel-24. Cells in suspension, containing mostly MoDC and residual melanoma cells, were harvested and underwent a purification step by magnetic cell sorting using the CD1c biotinilated mAb followed by anti-biotin microbeads. In FIG. 21A it is shown that MoDCs can be distinguished from melanoma cells after labeling with CD1a (to identify MoDCs) and NG2 (to identify melanoma cells). In FIG. 21, panel B it is shown the percentage of CD1a+MoDCs before (cell culture suspension) and after purification with CD1c (column eluate). As shown, regardless of the culture conditions (pretreatment with Salmonella or 100 Gy irradiation) isolation with CD1c magnetic beads results in ≧90% DC purification.

Immature Mo-DCs up-regulate co-activatory markers and cytokines after coculture with SL-infected melanoma cells.

An important pre-requisite for the correct activation of the immune response by DCs is the expression of costimulatory markers and release of cytokines. Since DCs are in contact with melanoma cells, it is important to show the proper activation state and the cytokines release required for T cell activation. As shown in FIG. 22, coincubation of melanoma cells with MoDCs induced the upregulation of activation markers and cytokine secretion only when tumor cells were infected with bacteria. Interestingly MoDCs produced all the cytokines (IL-12p70, IL 1b, TNF) that are required for an effective induction of Th1 T cells, the most powerful in anti-tumor immunity.

SL-infected melanoma cell line dictates the capacity of autologous Mo-DCs to reactivate metastatic melanoma derived Tumor Infiltrate Lymphocyte.

Authors then assessed whether DCs cocultured with infected melanoma cells are able to activate Tumor Infiltrating Lymphocytes (TILs). Their activation could be used to show an anti-melanoma antigen specific response. To proceed in this direction authors have generated, from fresh surgical sample, a melanoma cell line, as previous described. To generate TIL, the non-adherent fraction was cultured with high dose of IL-2 in order to expand T cell population. As show in FIG. 23, TIL secrete high level of IFN-γ in response to autologous Mo-DCs pre-cultured with autologous Salmonella-infected melanoma cell line. On the contrary TIL cultured with MoDCs alone or MoDCs pre-cultured with non-infected melanoma cell line produced similar but negligible levels of IFN-γ secretion, suggesting the absence of significant antigen specific T cell activation.

This data indicate that Salmonella infection of melanoma cell line dictate the capacity of autologous Mo-DCs to reactivate metastatic melanoma derived TIL.

MoDCs cocultured with SL-infected SK-Mel24 induce an anti-melanoma CTL-response in vitro and recognize epitopes shared among melanoma cells.

Mo-DCs generated and loaded as previously described were analyzed for their ability to induce in vitro melanoma specific CD8⁺ T cells.

From HLA-A2 positive healthy subject PBMCs were separated and CD8⁺ T cells were purified.

Mo-DCs obtained from the same donor were loaded with SL-infected SK-Mel24 (SL-SK), and cultured, with purified CD8⁺ T cells. Proliferating cells were expanded for 7-10 days in complete medium supplemented with 20 U/ml of rhIL-2. Cytotoxic properties of generated CTLs were tested against selected targets.

As shown in FIG. 24, CTLs induced by SL-SK loaded HLA-A 0201 matched semi-allogeneic Mo-DCs are able to lyse the HLA-restricted SK-Mel24 and a different melanoma cell line, but neither a class I negative melanoma cell line or a HLA-matched colon cancer cell line. Those data demonstrate that SL-infected SK-Mel24, as a “universal” donor of Melanoma Associated Antigen, can transfer antigens to MoDCs and generate CTLs able to recognize antigenic determinants shared among melanoma cells. Such antigen-specific cytotoxicity was dependent on gap junctions (FIG. 25A), because CTLs generated by Mo-DCs cocultured with SL-infected SK-Mel24 in presence of Heptanol, are unable to lyse the specific target, suggesting that, melanoma antigen trafficking through gap junctions is necessary to activate a CTL-response. Moreover, (FIG. 25B) the lytic activity is, also, paralleled by IFN-γ secretion. To further analyze the functionality of such CTLs authors, also, evaluated their ability to produce TNF-α and express CD107a (a marker of cells degranulation). The stimulation of CTLs with PMA-Ionomicin induces TNF-α production in CTLs generated with Mo-DCs cultured both with SK-Mel24 and SL-infected SK-Mel24, but, on the contrary, a larger number of degranulating CD8 T cells is only present when MoDC were cultured with SL-infected SK-Mel24 (FIG. 25C). This shows, again, the important role of SL-infected melanoma cells in the generation of cytolytic CD8 T cells.

### Discussion

Authors provide evidence that it is possible to exploit an immune response to infection that has not undergone tumor immune evasion pressure. In fact tumor cells that generally downregulate GJIC to lose control from the environment, upregulate Cx43 in response to Gram negative bacteria, i.e. Salmonella, or to bacterial components, such as LPS, flagellin or LTA, or gamma-IFN. The upregulation of Cx43 in tumor cells coincides with the generation of functional GJ both between tumor cells and between tumor cells and DCs. These newly formed GJs can be successfully exploited for the transfer of processed antigenic material to the DCs for T cell activation. This is a dominant pathway of cross-presentation that leads to the establishment of effective anti-tumor immunity in vivo. The requirement of preprocessing of antigens within the tumor cells guarantees that DCs will present a repertoire of peptides effectively presented by tumor cells. This feature was recently described also for the apoptosis-dependent cross-presentation pathway of viral infected cells (36). It is likely that also viruses may upregulate GJ proteins in infected cells and as it has been recently shown that also apoptotic cells can establish GJs for intercellular communication (14) it is possible that virus-infected apoptotic cells can allow transfer of antigenic material to acceptor cells. Through the use of infection of tumor cells authors have unveiled a mechanism that could be commonly employed during bacterial or viral infections that do not directly lead to apoptosis of the infected cells. Tumors develop several immune evasion strategies to promote the generation of more aggressive types with reduced immune responsiveness (37). Authors propose here that GJ downregulation could as well represent another mechanism of immune evasion occurring early during tumorigenesis. This would inhibit tumor antigen cross-presentation and prevent the development of anti-tumor immunity. The response to infection, however, should not undergo immune escape pressure in developing tumors and therefore be retained ‘intact’. Upon infection, tumors could naturally initiate the anti-infection program leading to Cx43 upregulation and GJIC, as described here. This cross-presentation pathway can be employed in vitro for the generation of very potent DC-based vaccines. In fact, authors have compared different pathways of tumor antigen cross-presentation like direct loading with tumor peptides, tumor lysates and apoptotic bodies. Authors found that the Cx43-dependent mechanism was the only one having a benefit in a therapeutic setting. In addition, when tested in a preventive setting that is the ideal situation in patients having undergone surgical resection of the tumor, vaccination with DCs loaded with infected tumor cells led to 100% of the mice free of tumor at the end of the experiment.

The strategy can be translated to other types of tumors, as authors have shown Cx43 upregulation also in other pathologies. Given the ability of DCs to also exchange material via GJ (35), this pathway could lead to the amplification of a specific response and could be more widely used than previously appreciated.

### Materials and Methods

Mice, Cells and Bacterial Strain

Five-week-old female C57/BL6J and OTI OVA-TCR transgenic mice were purchased from Charles River and maintained in Specific Pathogen Free animal house. Mouse studies were conducted according to the Italian law on approved experimental protocols.

The murine melanoma B16F10, B16F100VA (called throughout the paper B16 and B16-OVA, a kind gift from Dr. P. Dellabona (ATCC®, CRL-6475™), and B16BL6 were cultured in RPMI 1640 medium supplemented with 10% fetal bovine serum (FBS), 2 mM glutamine, 100 U/ml penicillin, 100 μg/ml streptomycin, 50 M 2-mercaptoethanol (complete RPMI). B16-OVA was cultured in the presence of 100 μg/ml Hygromycin B. The murine melanoma C57/B1 was grown in Minimum essential medium (MEM) supplemented as above plus 1% non essential amino acids (complete MEM). The murine dendritic cell line DC1 was generated in our laboratory following an established methodology (38). The human melanoma cell lines SK-Mel-24 is available at ATCC [SK-MEL-24 (ATCC®, HTB-71™)]. The human melanoma cell lines were cultured either in complete MEM supplemented with 1% sodium pyruvate [WM-115 (ATCC®, CRL-1675™), WM-266.4 (ECACC, 91061233), MEWO (ICLC, HTL97019), SK-MEL-31 (ATCC®, HTB-73™)], or in Dulbecco's MEM supplemented as above [ IGR-1 (DSMZ, ACC 236), IGR-37 (DSMZ, ACC 237), IGR-39 (DSMZ, ACC 239), RPMI-7951 (DSMZ, ACC 66) and CHL-1 (ATCC®, CRL-9446™)]. The T cell lymphoma Karpas-299 (DSMZ, ACC 31) and the acute promyelocytic leukemia NB4 (DSMZ, ACC 207) were cultured in complete RPMI, the prostate adenocarcinoma PC-3 (ATCC®, CRL-1435™) was cultured in complete Ham's F12, and lung carcinoma A549 (ATCC®, CCL185™) was cultured in complete DMEM. The colorectal adenocarcinoma Caco-2 (ATCC®, HTB-37™) was cultivated in complete IMDM.

Human Primary Melanoma Cell Lines.

Fresh melanoma specimens were processed to obtain a single cell suspension. Tissue specimens were cleaned with scissors and forceps to remove the fat (yellow part) and the connective tissue (white soft part). Tissues were cut into small pieces and digested in complete RPMI+ collagenase (1 ug/ul) and DNAse (10 u/ml) at 37° C. for 20 min. Cells were washed, counted and plated in RPMI 1640 medium supplemented with 10% fetal bovine serum (FBS), 2 mM glutamine, 100 U/ml penicillin, 100 ug/ml streptomycin, 50 uM 2-mercaptoethanol (complete RPMI). Initial successful culture was considered as the growth of cells up to passage 4. Salmonella typhimurium SL3261AT (received from Dr. Gordon Dougan) is an aroA-metabolically defective strain on SL1344 (received from Dr. Gordon Dougan) background and is grown at 37° C. in Lurian broth. Salmonella typhi Ty21a is a commercial strain called Vivotif Berna.

In vitro infection with bacteria and treatment with Pathogen-associated molecular patterns (PAMPs)

Single bacterial colonies were grown overnight and restarted the next day to reach an A_{600 nm}=0.6 corresponding to 0.6×10⁹ CFU/mL. Murine and human melanoma cells were incubated with bacteria for 2 h, at a ratio of 1:50 (cell/bacteria), in the appropriate medium without antibiotics. Cells were washed with PBS and incubated in medium supplemented with 50 μg/mL gentamicin for 12 or 24 h in order to kill extracellular bacteria, with or without IFN-g(100 U/ml).

B16 cells and DC1 DCs were plated in 6-well plates (2×10⁵ cells/well) and grown for 18 h. Cells were incubated with PAMPs in complete medium with or without IFN-g(100 U/ml) for 24 hours. The PAMPs used were LPS (1 μg/ml, Sigma), flagellin (0.1 μg/ml, Alexis) and LTA (10 μg/ml, Sigma).

Immunoblot Analysis

24 hour after infection or treatment with PAMPs, melanoma cells were scraped in ELB buffer (250 mM NaCl, 0.5% Nonidet P-40, 50 mM HEPES [pH 7.0], 5 mM EDTA) containing 1 mM dithiothreitol, 0.5 mM phenylmethylsulfonyl fluoride, and protease inhibitors. Cell lysates were run on SDS page and transferred on Immobilon PVDF membranes. Membranes were probed with a polyclonal rabbit-anti-Cx43 antibody (Sigma) or with a monoclonal mouse-anti-Cx43 antibody (Zymed) or with mouse-anti-vinculin antibody (Sigma) overnight at 4° C. and incubated with anti-mouse horseradish peroxidase-conjugated IgG (Calbiochem) or with anti-rabbit horseradish peroxidase-conjugated IgG (BioRad) antibodies for 1 h. Visualization was carried out with ECL (GE Healthcare). Bands were quantified by densitometry using NIH Image-based software Scion Image (Scion Corporation).

Real time PCR for connexin evaluation.

B16 cells were treated with Salmonella or PBS as a control, for 1.5 hours, extracellular bacteria were killed by the addition of gentamicin, and cells were incubated at 37° C. for additional 24 hours.

Total RNA was isolated from cells using RNeasy Mini Kits (Qiagen, Valencia, Calif., USA) and 1 μg RNA from each sample was reverse-transcribed using 200 U SuperScript II RT (Invitrogen, Carlsbad, Calif., USA) according to the manufacturers' protocols. One μg (50 ng) of cDNA was amplified by PCR using the SYBR Green PCR Master Mix (Applied Biosystem, Darmstadt, Germany) and specific primers of interest. Real-time PCR was carried out using a 7500 Real-Time PCR System (Applied Biosystem, Darmstadt, Germany) and the amplification was performed with 40 cycles of 15 s at 95° C. and 60 s at 59° C. To normalize each sample (run in triplicate) for total RNA content, control housekeeping gene (TBP) was used under similar PCR conditions. The relative expression level is expressed as fold change to untreated cells.

Immunofluorescence Analysis

B16 cells were plated onto coverslips (5×10⁴ cells/coverslip) and grown for 18 h. Cells were infected with Salmonella. After 24 hours the cells were fixed in 4% (w/v) paraformaldehyde in PBS for 15 min. Blocking and permeabilization were carried out incubating the cells in PBS, 3% (w/v) bovine serum albumin (BSA) and 0.1% (w/v) Triton X-100 for 20 min. The cells were then stained with polyclonal rabbit-anti-Cx43 primary antibody (Sigma, 1:2000) and with an anti-rabbit-Cy3-conjugated secondary antibody (Jackson ImmunoResearch laboratories, 1:400). Cell nuclei were stained with 4′,6-diamidino-2-phenylindole. Confocal microscopy was carried out using a Leica TCS-SP2 (Leica Microsystem).

ShRNA-Mediated Knockdown

To generate stable clones of B16 and B16-OVA cells silenced for Connexin 43, authors infected tumor cells with MISSION™ Lentiviral Transduction Particles specific for Cx43 (SIGMA), following the recommended protocol. Four different lentiviruses were tested of which three were successfully knocking down Cx43. One clone of successfully Cx43shRNA and one non-successful were generated by limiting dilution and were tested for silencing before and after bacterial infection. The unsuccessful clones were used as negative controls for the experiments (Ctrl-B16 and Ctrl-B16-OVA).

Mice Treatments

10⁵ B16 control cells (Ctrl-B16: non successfully silenced for Cx43) or 3×10⁵ Connexin 43-interfered B16 (Cx43shRNAB16) were subcutaneously injected in the left flank of C57/BL6J mice (day 0) and after 3 days 5×10⁴ B16 WT cells were subcutaneously (s.c.) injected in the right flank. At the days 11 and 15, 10⁸ CFU Salmonella or PBS as a control, were intratumorally injected in the left-flank tumors. The given dose of Salmonella was confirmed by plating bacterial dilutions on terrific broth agar plates. For the depletion of CD8⁺ T cells 100 μg of rat anti-CD8 antibody or isotype control antibody were injected in the peritoneum of the mice at the days 10, 14, 18, 21, 24, 27. Tumor growth was monitored measuring the two visible dimensions with a caliper every 2-3 days. Statistical significance at each time point was calculated as described below.

For therapeutic vaccination, 10⁵ B16 cells were s.c. injected in the left flank of C57/BL6J mice (day 0) and after 4 and 8 days (or 12 and 16 days in case of large tumors >0.4 cm² or 8 and 11 in other case) 3.5×10⁵ DC1 DCs were s.c. injected in the right flank.

For the combined vaccination, in some experiments the anti-CTLA4 or the isotype control was given intraperitoneally in the same days of the vaccine and at day 11 (100, 50 and 50 μg), while in other experiments the dacarbazine was administered intraperitoneally on day 8 for 4 days (5 mg/kg/day). For preventive vaccination, 3.5×10⁵ DC1 DCs were s.c. injected in the right flank of C57/BL6J mice (at days 0 and 4) and at day 4 (or day 21) 10⁵ B16 cells were s.c. injected in the left flank. The day before vaccination, Ctrl-B16,Cx43shRNAB16 or C57/B1 cells were infected in vitro with or without Salmonella, and then cocultured with DC1 DCs in a 1:1 ratio. After 24 hours DCs were purified by CD11c⁺ selection (MACS Milteny Biotec), and treated with 25 μg/ml Mitomycin C (Sigma) for 20 minutes at 37° C., to arrest the proliferation of possible remaining tumor cells. For the comparison experiment of different vaccination protocols, DCs were also incubated with either B16 lysate after 3 freezing and thawing cycles (B16 lysate), B16 UV-treated cells (B16 UV-treated) or with a mix of TRP2 and GP100 (1 mg/ml each peptide) peptide (TRP2/GP100).

Dye Transfer Assays

Microinjection assay. B16 cells were infected or not with Salmonella and after 24 hours 400 cells were microinjected under an inverted phase contrast microscope (axioverts100 Zeiss) with a mixture of 2% Lucifer yellow CH (457.25 Da, Sigma) and 1% tetramethylrodamine dextran (70 k Da, Molecular Probes) using an automated microinjection system at pressure of 1200 hectopascal applied for 0.2 sec. FACS assay murine cells. DCs were labeled with 10 μM of the dye 7-hydroxy-9H(I,3-dichloro-9,9-dimethylacridin-2-one) (DDAO, Molecular Probes) for 15 minutes at R.T. in the dark and then extensively washed with PBS. B16 cells were infected or not with Salmonella and after 24 hours were labeled with 0.5 μM calcein-acetoxymethylester (calcein-AM; Molecular Probes) in serum-free medium for 30 minutes at 37° C. and then co-cultured with DCs at a ratio of 2:1. Calcein transfer between tumor cells and DCs was evaluated by cytofluorimetry. The co-culture of B16/DCs was carried out in the presence or absence of (3.5 mM) Heptanol (Sigma).

FACS Assay Human Cells:

DCs were labeled with 1 μM of the dye 7-hydroxy-9H(I,3-dichloro-9,9-dimethylacridin-2-one) (DDAO, Molecular Probes) for 10 minutes at R.T. in the dark and then extensively washed with PBS+10% South American fetal bovine serum. Melanoma established or primary cell lines were infected or not with Salmonella and after 24 hours were labeled with 0.5 μM calcein-acetoxymethylester (calcein-AM; Molecular Probes) in serum-free medium for 20 minutes at 37° C.

Cells were co-cultured with DCs at a ratio of 1:1 for the indicated time points. Calcein transfer between tumor cells and DCs was evaluated by cytofluorimetry. To assess the GJ (CX43) dependent dye transfer the co-culture of Mel/DCs was carried out in the presence or absence of (3.5 mM) Heptanol (Sigma).

Confocal assay. B16 cells were infected or not with Salmonella and 24 hours later were stained with 3 μM calcein-AM in serum-free medium for 20 minutes at 37° C., while DCs, previously treated with LPS for 1 hour, were stained with 1μM DDAO for 10 minutes at R.T.. After the staining a drop of each population was plated in proximity to one another onto microscope slides and cells were co-incubated for 1 hour. Dye transfer was visualized by a Leica SP2 Visible Laser Confocal Microscope. The movie was generated using Imaris software.

In Vitro Cross-Presentation Assay

B16 and B16-OVA cells alone or together with DCs were treated or not with Salmonella and 24 hours later were stained with 5 μM of Carboxyfluorescein Succinimidyl ester (CF SE) for 20 minutes at 37° C. in the dark and then washed with cold PBS. Where stated, B16 and B16-OVA cells were treated with 10 μM Lactacystin (Sigma) overnight. Tumor cells were then added to DCs previously matured with LPS and IFN-g (1 μg/ml and 100 U/ml) in a 1:1 ratio and left in co-culture for 24 hours in the presence or absence of Heptanol (3.5 mM). After 24 hours cross-presentation of OVA peptide by DCs was analyzed by cytofluorimetry on CD11c⁺ cells using an antibody that recognizes the OVA SIINFEKL peptide in association with MHC I (anti K^{b}OVA, 25-D1.16).

Analysis of IFN-g in the Tumor Mass

B16 control and Cx43 shRNAB 16-bearing mice were intratumorally injected with Salmonella or PBS as a control and after 1, 3 and 7 days tumors were removed and smashed in 1 ml PBS containing 0.5% triton, incubated on ice for 1 hour and centrifuged at 13,000 rpm at 4° C. for 15 minutes. Supernatants were analyzed for the presence of IFN-gby ELISA (R&D System), according to manufacturer's instructions.

Analysis of IFN-g produced in vitro by OVA-specific CD8 T cells

B16-OVA control (ctrl-B16-OVA) and Connexin 43-interfered B 16-OVA (Cx43shRNAB16-OVA) were infected with Salmonella and were co-cultured with DCs previously matured with LPS and IFN-g(1 μg/ml and 100 U/ml) in a 1:1 ratio. After 24 hours CD11c⁺ DCs were purified (MACS Milteny Biotec) and treated with 25 μg/ml Mitomycin C for 20 minutes at 37° C., to arrest the proliferation of any remaining tumor cells. 2×10⁴ DCs were cultured with 2×10⁵ CD8 T cells purified from OTI mice, with and without 1 μM of OVA₂₅₇₋₂₆₄ peptide. After 48 hours culture supernatant was assessed for IFN-gby ELISA (R&D systems).

Statistical Analysis.

Student's paired t test was used to determine the statistical significance of the data. Significance was defined as *, p<0.05 (two-tailed test and two-sample equal variance parameters). Statistic calculations and Kaplan Meyer's survival curves were performed by JMP 5.1 software (SAS Cary, N.C., USA).

MODC-tumor cell coculture and analysis of surface activation markers.

MoDCs were cocultured with Mel-04 (primary melanoma cells), and after 24 h only cells in suspension, containing MoDCs, were harvested. Cells were stained with mAb to CD86, and CD83 in order to evaluate maturation/activation marker expression. Supernatants were analyzed for cytokines secretion (IL-12p70, IL-1b, TNF-a and IL-10) by BD Cytometric Bead Array (CBA).

Human MoDC purification from melanoma-DC coculture.

MoDCs were cocultured either with a melanoma primary cell line (mel04) or SK-Mel-24.

Cells in suspension, containing mostly MoDC and residual melanoma cells, were harvested and subjected to a purification step by magnetic cell sorting. Briefly, cell suspension was labeled with CD1c biotinilated mAb followed by anti-biotin microbeads (Miltenyi Biotech). Labeled cell were loaded onto a magnetic column allowing only the retention of cell labeled by CD1c mAb (MoDC), the flow-through contains mainly melanoma cell (CD1c negative).

Melanoma Derived TIL Expansion and Activation.

TIL were obtained by culture non adherent single cell suspension, used to generate the melanoma cell line, in the presence of 5000 U/ml of rhIL-2 for 1 week. Patient-derived MoDCs were cocultured with infected or non-infected autologous melanoma primary cell line, generated as previously described.

TIL were cultured with Mo-DC at 1:10 ratio and after 72h, supernatant was harvested and IFN-γsecretion was measured by specific human IFN-g ELISA (BD Bioscience), according to manufacturer's instructions.

CTL Generation and Functional Assays

Purified CD8 T cells by magnetic cell sorting were cultured with MoDCs previously cocultured with infected or non-infected Sk-Mel24 in the presence or absence of 3,5 mM heptanol. After 24-48h cultures were supplemented with 20 U/ml of rhIL-2 and after 7-10 days CTLs were either subjected to a second round of stimulation or assayed for their cytotoxic activity. Melanoma specific cytotoxicity was measured by using DELFIA cytotoxicity Kit (Perkin Elmer) and lysosomal-associated membrane protein-1 (CD107a) mobilization was performed by using BD FastImmune CD107a reagent (BD Bioscience), according to manufacturer's instructions.

Flow sheet of immunotherapy formulation (non-limitative example).

First round of immunotherapy treatment using a ‘Universal’ tumor cell line (SK-Mel-24 or a combination of tumor cell lines per specificity of antigen repertoire).

Day 0: Leukapheresis from patient

Day 0: Expansion of tumor cells

Day 0: Generation of DCs from Monocytes with GM-CSF and IL-4. Peripheral blood mononuclear cells (PBMC) are isolated via Ficoll density gradient. The PBMC (1200×10⁶) are loaded via bottles and tube connector or directly by pipette into one double tray cell factory. After 45 min to 1 h, the adherence is controlled by microscopy. The loosely adherent cells are mobilized by tapping the cell factories five times from either side, the nonadherent fraction is discarded. The cell factories are washed twice with pure, warm RPMI 1640 and 240 ml of complete medium without cytokines.

Day 1: Complete medium supplemented with (recombinant human) r-hu GM-CSF (final concentration 800 U/ml) and r-hu IL-4 (final concentration 500 U/ml) is added and carefully equilibrated.

Days 3 and 5: 40 ml of culture medium with GM-CSF and IL-4 in the same final concentrations are added.

Day 6: Irradiation of tumor cells, treatment with Salmonella Ty21A (ratio 50 CFU: 1 Tumor cell) for two hrs in medium without antibiotics and then for 2-3 h in medium with gentamycine and with patient MoDCs. Alternatively, the incubation of irradiated tumor cells, bacteria and DCs could also be carried out simultaneously.

Day 7: Non-adherent cells are harvested and prepared for generation of immunotherapy formulation aliquots by freezing.

Quality control of immunotherapy formulation. Generated DCs are evaluated by cytometry for assessment of purity and maturation, by microscopy to verify a mature morphology and phenotype, by a “washout test” (24 h in medium without cytokines) to determine stability and survival, and by allogeneic mixed lymphocyte reaction (MLR) to examine their stimulatory capacity.

Second Round of Immunotherapy Treatment Using an Autologous Tumor Cell Line

Day 0 specimen received from the surgery

Day 0 tissue is processed to obtain a single cell suspension after mechanical digestion and released cells are cultured.

Day 7-to generation of cell line: medium is changed when needed and cells are passaged to obtain a cell line.

MoDCs are generated and loaded as above.

Patient Treatment

Patients will be treated intradermally with 1-20 millions of DCs.

2 inoculation per month for 2 months

4 inoculation for the subsequent 4 months

(8 inoculations total)

### References

1. R. M. Steinman, J. Banchereau, Nature 449, 419 (Sep. 27, 2007).
2. P. van der Bruggen, B. J. Van den Eynde, Curr Opin Immunol 18, 98 (Feb, 2006).
3. A. Paschen, S. Eichmuller, D. Schadendorf, Cancer Immunol Immunother 53, 196 (Mar, 2004).
4. L. Chapatte et al., Cancer Res 66, 5461 (May 15, 2006).
5. B. J. Nicholson, J Cell Sci 116, 4479 (Nov. 15, 2003).
6. G. Mese, G. Richard, T. W. White, J Invest Dermatol 127, 2516 (Nov, 2007).
7. J. Neijssen, B. Pang, J. Neefjes, Prog Biophys Mol Biol 94, 207 (May-Jun, 2007).
8. T. J. King, J. S. Bertram, Biochim Biophys Acta 1719, 146 (Dec. 20, 2005).
9. M. Mesnil, S. Crespin, J. L. Avanzo, M. L. Zaidan-Dagli, Biochim Biophys Acta 1719, 125 (Dec. 20, 2005).
10. T. Bopp et al., J Exp Med 204, 1303 (Jun. 11, 2007).
11. E. Oviedo-Orta, W. Howard Evans, Biochim Biophys Acta 1662, 102 (Mar. 23, 2004).
12. H. Matsue et al., J Immunol 176, 181 (Jan. 1, 2006).
13. J. Neijssen et al., Nature 434, 83 (Mar. 3, 2005).
14. B. Pang et al., J Immunol 183, 1083 (Jul. 15, 2009).
15. H. Benlalam et al., J Immunol 182, 2654 (Mar. 1, 2009).
16. D. Bermudes, L. M. Zheng, I. C. King, Curr Opin Drug Discov Devel 5, 194 (Mar, 2002).
17. J. M. Pawelek, K. B. Low, D. Bermudes, Cancer Res 57, 4537 (1997).
18. M. Loeffler, G. Le'Negrate, M. Krajewska, J. C. Reed, Cancer Gene Ther 15, 787 (Dec, 2008).
19. M. Loeffler, G. Le'Negrate, M. Krajewska, J. C. Reed, Cancer Immunol Immunother 58, 769 (May, 2009).
20. J. Fensterle et al., Cancer Gene Ther 15, 85 (Feb, 2008).
21. U. Pertl et al., Blood 101, 649 (Jan. 15, 2003).
22. A. G. Niethammer et al., Cancer Res 61, 6178 (Aug. 15, 2001).
23. F. Avogadri et al., Cancer Res 65, 3920 (May 1, 2005, 2005).
24. F. Avogadri et al., Eur J Immunol 38, 1937 (Jul, 2008).
25. N. K. Haass, K. S. Smalley, M. Herlyn, J Mol Histol 35, 309 (Mar, 2004).
26. C. J. Wei, R. Francis, X. Xu, C. W. Lo, J. Biol. Chem. 280, 19925 (May 20, 2005).
27. A. Porgador, J. W. Yewdell, Y. Deng, J. R. Bennink, R. N. Germain, Immunity 6, 715 (1997).
28. G. Fenteany et al., Science 268, 726 (May 5, 1995).
29. G. Rotta et al., J Exp Med 198, 1253 (Oct. 20, 2003).
30. G. Rotta et al., J Exp Med 205, 657 (Mar. 17, 2008).
31. T. A. Groothuis, J. Neefjes, J Exp Med 202, 1313 (Nov. 21, 2005).
32. M. L. Albert, B. Sauter, N. Bhardwaj, Nature 392, 86 (1998).
33. A. L. Ackerman, C. Kyritsis, R. Tampe, P. Cresswell, Nat Immunol 6, 107 (Jan, 2005).
34. J. Banchereau, A. K. Palucka, Nat Rev Immunol 5, 296 (Apr, 2005).
35. A. Mendoza-Naranjo et al., J Immunol 178, 6949 (Jun. 1, 2007).
36. N. E. Blachere, R. B. Darnell, M. L. Albert, PLoS Biology 3, e185 (Jun. 1, 2005, 2005).
37. G. P. Dunn, A. T. Bruce, H. Ikeda, L. J. Old, R. D. Schreiber, Nat Immunol 3, 991 (Nov, 2002).
38. C. Winzler et al., J. Exp. Med. 185, 317 (1997).

1. Method for obtaining a specific tumor antigen peptide repertoire loaded and/or activated dendritic cell comprising the steps of:
a) exposing in suitable conditions a tumor cell expressing said specific tumor antigen peptide repertoire to at least one Pattern Recognition Receptor (PRR) agonist and/or to one inflammatory cytokine to obtain a tumor cell with an increased activity of at least one protein belonging to the group of connexins; and
b) co-culturing said tumor cell with an increased activity of at least one protein belonging to the group of connexins with dendritic cells, to get specific tumor antigen peptide repertoire loaded and/or activated dendritic cells;
wherein steps a) and b) are performed simultaneously or in sequence.

2. The method according to claim 1 wherein the group of connexins is selected from the group consisting of connexin 43, connexin 40, connexin 45, connexin 47, and connexin 50.

3. The method according to claim 1 wherein the inflammatory cytokine is gamma-IFN.

4. The method according to claim 1 further comprising step c) purifying said specific tumor antigen peptide repertoire loaded and/or activated dendritic cells.

5. The method according to claim 1, wherein the tumor cell is an established tumor cell line, or a combination of tumor cell lines expressing a specific tumor antigen peptide repertoire or a tumor cell isolated by a tumor affected subject.

6. The method according to claim 1, wherein the tumor cell derives from solid or non-solid tumors, including melanoma, lung carcinoma, colorectal adenocarcinoma, prostate adenocarcinoma, leukemia and T cell-lymphoma and the said specific tumor antigen peptide repertoire is specific for said tumor.

7. The method according to claim 1 wherein dendritic cells are autologous or HLA-compatible or -semi-compatible allogenic dendritic cells.

8. The method according to claim 1, wherein the PRR agonists are Gram-negative, Gram-positive bacteria or components thereof

9. The method according to claim 8, wherein Gram negative bacteria belongs to the Salmonella genus.

10. The method according to claim 9, wherein bacteria belongs to non virulent strains of Salmonella genus.

11. The method according to claims 8 wherein Gram negative bacteria components are LPS and/or flagellin.

12. The method according to claim 8 wherein Gram positive bacteria components are Lipoteichoic acid (LTA).

13. A specific tumor antigen peptide repertoire loaded and/or activated dendritic cell obtainable according to the method of claim 1.

14. A method of treating or preventing tumors comprising the administration of the specific tumor antigen peptide repertoire loaded and/or activated dendritic cell of claim 13 to a patient in need thereof.

15. (canceled)

16. A combination comprising the specific tumor antigen peptide repertoire loaded and/or activated dendritic cell of claim 13 antibodies and/or chemotherapeutics.

17. Method for generating specific CTLs comprising incubating a specific tumor antigen peptide repertoire loaded and/or activated dendritic cell of claim 13 with a sample of CD8+T cells isolated from a donor.

18. Method for increasing the activity of at least one protein belonging to the group consisting of connexins in a tumor cell expressing a specific tumor antigen peptide repertoire comprising exposing said tumor cell to at least one PRR agonist and/or one inflammatory cytokine.
